SEQUENCE LISTING (1 sequence)

SEQ ID NO: 1 - DNA, 9669 bp, Artificial Sequence
  Seed gene which is modified or mutated such that seeds exhibit lower levels of AGPase or lower levels of activity of AGPase.
tgttttttgtgtgtgaataaacttgttgccaataaagcgaagagcatatgtagtacgccaaaaactttacagcttgtcacatgcgaactaatttcgtcgcacatggatattcatgtgctcttttttgtacgtgcatatacttccttcgcctataaataaaagaagagtttccttatgacttcaaaagtgaactcacacatcactcaatatctatatccttccattttatatccctcggtgatggatgtacctttggcatctaaagttcccttgccctccccttccaagcatgaacaatgcaacgtttatagtcataagagctcatcgaagcatgcagatctcaatccccatgctattgatgtaagtggtgctatcttaactatgattttcgttttctgttccatctttgagtatcatatggagtaatattatttttaggaagtcttaggaaaggcttctttggggcagcttcaagcataattaaaagacactccagagccacatcatcacatgcatgcatatacaacacaacacaacacatgaagtgagcgatatctttttagtttttcgaactccaatttttttctcttcaataaaaaatacgattaaaaatccattctcatcattaaatctgcttccaccagatttttaaaactacatcgcatattgataagtttcaacgatattttttgggccaaaagttatcatgatgcttaccctcaagttaacatagtgtttacactaaagttgtcatgatatgttttatatataattttctcaagattttaaagctaccatggaacatgacaaatttaagtaattcaccatgacaattttaatttatggttcacgtcaaaattaagtcattgactatgcatttttaaagtaatttaacgacatattttgttttagttatgaaccgtgccaaaaatatttcatggatcatggcaatttttcataattcaccatgataagttttagtttcttatttttttataacatgacaaaattactttaaatgtagaagaaaaaattgttaaaatatatcatgataactttagtgtaaaaacaatttatgtgcaacaaacatgacagcttttaaccccaaaaagccattgaaacatatgaaatctagtttcagaaatctcgttgtgacgaatttaatgatgaaaatatgttttcaatcggatttttaatttaaaagataaatcattttaaaagctaaatttttaaaaaaaatattgccatcattagtttcatcatttatgcatacatgcgatgatatagtgtgatgtgtgcggatgaattcgttcggtcgatgatctccccgattgaatgtgtgaacgctatcagagtctttatcttccactgttctatcttatatattacgttttttttaaaattattgtcttaaatttatctagctacaaatatatctaaccttaaaacacgattagatacatccgttagataaatccatgataatttttttaagacggagagtatgtgctatgcctgtcaagcagaaaaatctgaaaagacaattaagagagaaagaagtttaccattgatattccaaaatcatcgtggctatgtactcctcaaggaggtcattccaaaagtgccgctgctcacgatctctctcactcccacgcagctctctctaaaagaaaaatggcaaaaaactgaaaatggaaagatcttacgaaaagattagttaaatttactcagccacactgcaccactcggtgtcaggcgtatctctctcccttacccctcgtgatctctcgccacgggagccccgtgactcgagctcgtcatccacctcaatggcgatggccgcggccgcctccccttccaagatcctgatccctccgcaccgagcctccgccgtgaccgctgccgcgtccacctcctgcgactccctccgcctcctctgcgcgccacgaggacggccaggcccgcgcgggttggtcgcgcgtccggttccgcggcggcccttcttcttctccccacgtgccgtgtcagactccaagagctcccagacttgtctcgaccccgacgcaagcacggtacgccgcctcgcctagccaaatgcggcgcttcttggccgcctagtcttgtctcgctgccctgatccgttgcgtccgtatctttccggatgagaatttgacacatgcgggaagttattgcctcggtaatttagatgcgcaaatgtggttcgcgtcttgtgttctcatgtggacatttcttagagatgataacaaaaaatactactctactttgctactagcgcatagcatgaacttttgactaatcatgtggacattttcatttgctccaattatttatttgtactagatttcagtaaatgtggcaactgtgggcgtttgttgtacagtcctacttaatcaattggtgtgggtctgacacgtgtgagccccataagaaatttataaagaagatggcatgatccatgacatgtggctcttaaagaccactgggcaatcagatcaagttcttggatttttatttgtaacttattcagttttcttctttgagttttgcttcagtacaccctttataaaaacattacgattttggatgtggtggacattaaaacttaccctttcattttaattaaaagggtagtagagtctattccacgtggtgcaaatcaatggtggttgcctctcttcctcacaaaccgttctggcatcaacacaactaaacaaagtaatacaaccaggcgagttttaggcgagataatagattggggtcttctgtgctggtgaagactctacgtgatgtgaaaaagttatacacaacaggaataacttggatcacatctcagctgcaatgctgattgagacatctgacgtccaattaaacccatattcggaagaaaaaactaaacataagttccagcttgattgataaataaaaggtcagaactattcaccagccagatgcccagtatctacaaaagattggtagacattgtagcttcagtttattggataaacttgttgccccatgtcacattcattccatgatctcttttggtgataaacaagctgaactcagtgccaaccgtctggaacacttgtttcttcgttctttgtttgatttactttgcaataggcaattgatgagtttctgctgtttgtgcagagtgttctcggtatcattcttggaggtggtgcagggactagattgtatcccctgacgaagaagcgtgcaaagcctgcagtgccattgggtgccaactacaggcttattgatattcctgtcagtaattgtctgaacagcaacatatcaaagatctatgtgcttacacagttcaactcagcttctcttaatcgtcatctctcacgagcctatgggagcaacattggaggttacamgaatgaaggatttgttgaagtccttgctgcacagcagagcccagataaccctgactggttccaggtatctcattcattgttatttaagtgtttttgtttaatgtgaaatgcgagattcatctactgatgaacatcataatttgtctcatgttagcatttagaagaaggcaaaatctataattccttcataagtactcgtgattgtatcatttcaccctctgtggaaatcccagggccagccttccaagaaccagaatagaaaagagacaatctgttccaagacgtcattgatattcctttttacagaaccttgatgtagattataagaattattatttggatactgccctaatagtcctctatttattatttccgattttctaaataattcaatttaatagcatgctatcacaccacagttttaagctcaagtagagatgctcagaaattttcatgaattgattttaacagtgtttctgaattatacgaatctgttttgcgtaccaagatctggtcctgaacaagttcactagttgcaaattttgaattagtatacgtgaatggtcagtgatgtaactttgattttgattcttatgagcattagccagtcatcatcatttataagtaaacacagcagatcaaactatgtttcatactttcgtatgtttgccgttataataatactattcatcatagcttctgctttagattgcgagtgctataccacacagctacatgcagtttctgctattttatgtcaaatcagttaccctacagcgtttttctagataataagaaccaaagtcatgtccgtgaggacttgaacctgggtggctgggctgtagatccactcccctaacaaagtgagctctgctcacttcttgataatcataaactacataaagtgttgctagggtcccatgcaagcttttgtagggtattcactttgtcctatcatcttacctcagggtactgcagatgctgtaaggcagtacttgtggctattcgaggagcataatgttatggagtatctaattcttgctggagatcacctgtaccgaatggactatgaaaagtttattcaggcacacagagaaacggatgctgatattactgttgctgccttgcccatggatgaggaacgtgcaactgcatttggccttatgaaaatcgatgaagaagggaggataattgaattcgcagagaaaccaaaaggagaacagttgaaagctatgatggtacactgacactgtgcctttctaactaatttcagatatacagttgtgaaccatcattcattacaccacaaaatctcttctgttgaatgcatttacaccatgttgctacctgttttggtcttgtaatggtacactggcgctgtgcctttctaactaatttcagatatacagttgtgaaccatcatttattacaccaaaaatctcttgtgttgaatgcatttacaccatgttgctgcctgttttgatcttgtaggttgatacgaccatacttggccttgaagatgcgagggcaaaggaaatgccttatattgctagcatgggtatctatgttattagcaaacatgtgatgcttcagcttctccgtgagcaatttcctggagctaatgacttcggaagtgaagttattcctggtgcaactagcactggcatgagggtaggcaaagctcattgagttagtagttttttttcgctgcttctgcttttatgatttgaatcattttagcctcagagaaactgtcaagtcatatgtttatcgttcggaaagggatacaataggttattggatatgcactttgtagaaacgggagggggagaggactacctccagatgggtcatgggtgttgtggatgtgtggcggctggctcactcgggaggactggaaacacctccttctaggtcatctcaagggctaggccttccgggcttaagtgagatgggccatacagcccataccggttcaacactccccctcaagatgggtggtagatatctagcatttcgatcttgtaacatgccaagttacagtcctttgttcccagtccctttgtcaagcaatctgcgaactgttgccacaagtttcagcacacacccgcgtgcaatccacttgcaccctgactcgatgcaagccgcatcatagagttccaaaggatcacaattatcctgctccgcccacaaagcctgcaactctgccacatatgccatcacggacatgtcgtcaccttggcgcaaccgactgatcttcccctcaatctgagcgattagcatgaaattacccttgcctgagtattgggtggatagggtcttccatatctcggagtggatagtccctccacagagcgtccaatggagggcaccactgagttcaacaaccagccaacgagcacggagcttatgaccttccacctctttccctccgcggtattcctgtctcctggttcatcaatcgtatccagtaagtgcccatcaagttccttctgttctacggtcagcaatgccctcctggaccaactctagtaatttgtggccccctccagcttcatgtccaggggtgacatttcgagcttctgagccacttcctgtcgaggaacgatggccccagagttggactctgcgaggatcttagcgagcttctcgaaagcctcagcaagcgcatttggttcagccatctccgatccggtaatcagcaacagcagccctcagcagcacagccctacagctgcacggttggtccctttacgccccctggctagcagcagcacccagcagctccaagtgcaagcagcaaccacagcctcttcctccagcagcagttctcttccttcttcctctgcaaacagcagcagttctcttcctccttcctctgcaaacagcagcagttccagaggatcttggcctccaacagcagccaacccccgcagcagttctccagctcccggtcccctgcagacacacgcacacacagcagcacaacaaggagctgcccttctccactatccttctcctcttctcctcccgcagcaccacagcccaccacgctgaggagcaagcagcagccacagcacaacccactctcggggagaccaagccgctcctcagcttcctcctcctgccgtgcagcacagcagctccacctctccttcttcctctgccgcgcagcacagcagctccacctctccttcctctccaacgagccgcacctcctgcgcagccacaccgtgcctcccagatccgccgctgcctcaccggacgccgcccaaatccgccgccacctcgctggacgtcgccgccgtcttctcaggccccgccgtcgctggcctctgataccatgtagaaacgggagggggagaggactacctccagatgggtcatgggtgttgtggatgtgtggcggctggctcactcgggaggactgggaacacctccttctaggccatctcaagggctgggccttgtgggcttaagtgagatgggccatacagcccataccagttcaacacacttccattggcattcatagttgtgatatgtgcttcttaagagttttgttattgttgccgacaggtacaagcatacctatacgacggttactgggaagatattggtacaattgaggcattctataatgcaaatttgggaattaccaaaaaaccaatacctgatttcaggtgcgctttcattttttgccttgttgtggacaaatattatgaaattgcatgcatgtaaagtgttagaattgtcccctattgatttaatgtatacgttcaatttgaattcagtttctatgaccgttctgctcccatttacacacaacctcgacacttgcctccttcaaaggttcttgatgctgatgtgacagacagtgtaattggtgaaggatgtgttattaaagtaagtagcctttttcagttggctctcggtatgctaacccttcttcaggtgttccatttcgtgctaacaaaccttaagcttttaaagacatatttcaaaaccatctatacttctttatgggctgtgattgttatatcttctctcaagtgatttttgatgctgtgtgttataaagacttctaagttacatttgcctttctttgctctccacgtagaactgcaagatacaccattcagtagttggactccgttcctgcatatctgaaggtgcaataatagaggacacgttgctaatgggtgcggactactatgaggtaaaatcagacaggtgtaatatgcttctgccaaagtgatgtactcaccccttcttttattgttcaacagactgaagctgataagaaactccttgctgaaaaaggtggcattcccattggtattggaaagaattcacacatcaaaagagcaatcattgacaagaatgctcgtattggagataacgtgatggtatgccatattgatatacttatgcttaaacatctattggtttctctttttcttttccactgtggtaggaaccgctaaggttctaccgggtctagggcggaggttgtaggggatgaagcggagttggcgagggctgtctcgcggcggccggcggcgggacgccgttgcgcgcgagagggaggcggcggcggtggaaggcggcagcgcagctagggttccggctcctctgggagccgggcaatagagttatgactatattgcttaattcccaaaagagttgtttacattggtttatataatctcgataacttggactctaagatcactaagataacttggactctaagataacttggacactaagataactaagataacatgggctaagcccgtaactaatcctgcccattgggcctcctccgttggttcgtagtaccggtcataacacactgcaacattgtcatgctgaatatgtaacttgaacataacttttctcacggtaatgtccaaaatgtaaccattatataacaagctttaggtcttgtcgggttcataggaaaagtgagaaaaatgtaggaagaggatattttcttttagcgtcactgttcattcgttttgttcaaaggagaagtggaggaaagttcctttgatcacacttcgaaaggaaaatcgcaggaattttataatctacttgacttccgtcttagttatccttcttcatgtgctttgactttgatttgactgtcattagcggatggttaagacatgctgataatgtcaagggaggtcggggtagaccaaacttgacatgggaggagtctctaaagagtgacctgaagaactggaatatcaccaaagatttagccatggagaggggtgtgtggaagttagttattcactgccagaaccatgacttggttttgatatcggatggatttcaactctagcctaccccaacttgtttgggactgaaaggctttgctgttgttgtcctatgttctattccttagaagcagacttatattagggtgaaaacttgttttgcacttccattcctaccctcttttttgtcattttctttctatttctatgttatcataatcctgtgaaccaaacatatcctatattgtatatccatttccttgaacatgatatcacgcactgtgcgttgtttttggtagtgatctggactcattggtatattgtagataatcaatgttgacaatgttcaagaagcggcgagggagacagatggatacttcatcaaaagtggcatcgtaactgtgatcaaggatgctttactccctagtggaacagtcatatgaagtaagttgtctcctcgtacacacctcggtgtctgcaatcagttatgttttattttagaaactatgaacatgttgtaaaccaaaaatgatgcaaatgcagcaatacagttggtacatgcaaaccatgcactggtatcctatacattcaatttgagattttagcactcttcttgtaagtagttgactctgtttgggttgccctgcaggcagatgtgaaatgtatgccaaaagacagggctacttgcgtcagtctggaatcaaccaacaaggccgcgaagagatcataaaataaaaaggagtgccatgcgagtcacttctacacccttttccccccttgatgtattaggaactgtgatgtacaagcaactgtgatgcacttacgcgaagtgcccctggattcagctttctctttgcttgtaactggtttccagcagaccatgctatttgttgtatggttcgtgcaaaaccttgcgatgctttatatatgctttatatataaaacaagatgaatcccgcgcgttgctgcgg

(19) United States
(12) Patent Application Publication
(10) Pub. No.: US 2006/0064780 A1
(43) Pub. Date: Mar. 23, 2006

(54) Method and product

(75) Inventors: Lars Munck, Fredensberg C (DK); Birthe Moller, Fredensberg C (DK); Hilde-Gunn Opsahl Sorteberg, As (NO); Heidi Rudi, As (NO)

Correspondence Address: SEED INTELLECTUAL PROPERTY LAW GROUP PLLC, 701 FIFTH AVE, SUITE 6300, SEATTLE, WA, 98104-7092, US

(73) Assignee: Norges Landbrukshogskole, As (NO)

(21) Appl. No.: 11/180,988

(22) Filed: Jul. 12, 2005

Related U.S. Application Data
(60) Provisional application No. 60/592,091, filed on Jul. 28, 2004.
(60) Provisional application No. 60/673,866, filed on Apr. 22, 2005.

(30) Foreign Application Priority Data
Jul. 12, 2004 (GB) GB 0415556.0

(51) Int. Cl. A01H 1/00; B02C 17/00

(52) U.S. Cl. 800/284; 241/24.1

(57) ABSTRACT

The present invention relates to seed products from seeds having low starch (e.g., 25-40%) and high β-glucan (e.g., 10-25%), particularly from seeds with moderate amylose levels, particularly from mutated seeds (e.g., in which the AGPase gene is mutated), methods of preparing such products, methods for screening for seeds, β-glucan preparations and novel seeds having low starch and high β-glucan.

### CROSS-REFERENCES TO RELATED APPLICATIONS

This application claims the benefit under 35 U.S.C. § 119(e) of U.S. Provisional Patent Application No. 60/592,091, filed Jul. 28, 2004, and U.S. Provisional Patent Application No. 60/673,866, filed Apr. 22, 2005; and the benefit under 35 U.S.C. § 119(a) of Great Britain Patent Application No. GB 0415556.0, filed Jul. 12, 2004, where these applications are incorporated herein by reference in their entireties.

### BACKGROUND OF THE INVENTION

1. Field of the Invention

The present invention relates to seeds which have low starch and high β-glucan levels, preferably mutant seeds genotypically altered relative to wild-type (for example by mutation of a gene encoding a component of AGPase), methods of producing the same and screening for the same and methods of producing seed (e.g., grain) products from said seeds as well as methods of producing those products.

2. Description of the Related Art

Consumers place increasing importance on obtaining wholesome and nutritious ingredients with desirable health properties. For agricultural crops this must be matched by plant varieties suited to production in different conditions, environmental concerns, and climate that can deliver an appropriate harvest. For example, in cereals and potato tubers, which together represent more than half of the total land in the Nordic region in cultivation, it is the properties of the storage proteins and the carbohydrates, particularly starch, that are critical both to the final product and to the processing industry.

There is evidence that insulin resistant syndrome (IRS), and therefore diabetes and cardiovascular disease, can be prevented by a high fiber/low glycemic index diet (Ludwig, (2003) Asia Pac. J. Clin. Nutr. 12, Suppl:S4). β-glucan is of major importance in cereals for food because of its properties in human nutrition as a dietary fibre for regulating the flow in the digestive system, lowering the cholesterol value in the blood and reducing the risk for colon cancer (McIntosh et al. (1991) Am. J. Clin. Nutr. 53, 1205-1209). Since the human body is largely unable to utilise β-glucan as an energy resource, the energy value of a cereal product high in β-glucan is correspondingly reduced implying a decrease in blood glucose as expressed in the glycemic index (GI), of great importance in diabetes prevention and treatment (Salmeron et al. (1997) Diabetes Care 20, 545-550), as well as to counter obesity.

1,3-β-glucan (1,3-β-G) constitutes one of the structural macromolecules in the cell wall of higher plants (wound-induced sugar on the plant new cell wall, callose). Various structures of 1,3-β-glucan are found; some are composed of a 1,3-β-linked glycolytic backbone and exist as a single-chain linear conformer such as pachyman, curdlan, paramylon, and laminarin, whereas some from fungal sources belong to diverse shapes of 1,3-β-glucan main chain with different degrees of 1,6-β-glucan branches and length such as lentinan, schizophyllan, and yeast glucan. Cereal 1,3-β-glucans are mixed linked glucans (MLG; oat and barley glucan), which contain not only the above two linkages together but also intramolecular 1,4-β-glycosyl linkages.

The health functions of 1,3-β-glucan have attracted much attention in recent years. Besides being a source of dietary fiber, it is linked with certain biomedical effects such as host defence potentiator (HDP), antitumor, anti-infective, and immunostimulator effects. Well-identified foods containing 1,3-β-glucan with biological functions are cereals, yeasts, and fungi (mushrooms). Oat bran and barley MLGs are considered to be health foods that have proven to reduce total cholesterol and low-density lipoprotein (LDL) levels of hypercholesterolemia patients (Ko and Lin (2004), J. Agric. Food Chem. 52, 3313-3318).

A low starch, high β-glucan seed would therefore be an attractive prospect to address these health issues. Such seeds having modest amylose levels have however not as yet been identified.

Targeted improvement of the starch, β-glucan and protein components so that they are suited to specific feed, food, and non-food applications nevertheless requires integrated research extending from the many thousands of genes potentially involved in determining quality to the actual carbohydrate and protein structures produced in the seed, e.g., grain. This is because starch biosynthesis is a flexible process involving multiple feedback points, ultimately resulting in the complex three-dimensional amylose and amylopectin molecules with particular thermodynamic properties. In addition, protein and β-glucan deposition in grains is linked to that of starch. Finally, at the phenome (the collection of phenotypes of a cell or organism) level, and for various cereal applications, the different biopolymers interact cooperatively to give the final phenotype and functionality needed by industry and sought by consumers.

Barley (Hordeum vulgare L.) is a true diploid closely related to wheat and rye; it is one of the most important crop species in the world. It is the most cultivated crop in Sweden, Norway, Denmark and Finland. Globally, it is the fourth-most cultivated cereal crop (after wheat, maize and rice) and is grown (2002) on 54 Mha with an annual yield of 132 Mt (www.fao.org/WAICENT/), and 1.9 Mha and 9 Mt respectively for the Nordic countries.

Barley grain is largely used as animal feed and malt and, to a lesser extent, as food. It is an excellent energy source because the grain consists of 80% carbohydrates, mostly starch; in many countries in Africa and Asia barley is an important part of the diet. The soluble fiber of barley makes it a good “functional food” and for this reason it has been rediscovered in Europe. The plant is remarkably plastic in its adaptation to altitude, latitude, soil moisture and salinity, and temperature.

Its wide geographic distribution has led to a vast array of genetic variability in the germplasm, stored in collections such as the Nordic Gene Bank (Alnarp), and the Risø and Carlsberg barley collections, most of which remains to be characterized and exploited (Falk et al. (2001) In: Progr. Botany, e. Karl Esser, ed.: Springer-Verlag, Berlin), pp. 32-50.). A large collection of barley mutants affecting the endosperm phenotype is available and constitutes an enormous potential resource, hitherto almost unexploited, for crop improvement given appropriate molecular markers to prevent gene drag in the crosses. Many of these mutants have unknown effects on the metabolome (all pools of metabolites in a cell), which includes carbohydrate and storage protein biopolymers.

The cereal endosperm is our largest single primary food source, and thus among the most economically important structures in biology. It consists of two tissues, the interior starch-filled endosperm and the outer epidermal layer called the aleurone. The barley aleurone layer also harbors most of the grain phosphate, which is deposited as phytin particles in protein bodies (Falk et al., 2001, supra). Development of the endosperm is orchestrated by the coordinated activities of a large number of genes that encode metabolic and regulatory enzymes and other proteins (Becraft et al. (2000), Developmental biology of endosperm development. Kluwer Academic Publisher, Dordrecht, The Netherlands; Olsen (2001), Annu. Rev. Plant Physiol. Plant Mol. Biol. 52, 233-267; Olsen (2004), Plant Cell 16, S214-S227; Olsen et al. (1998), Trends Plant Sci. 3, 168-169).

The starch-filled cells in the interior of the barley endosperm become densely packed with starch granules and hordein protein bodies. β-glucan also becomes deposited as an essential part of the barley grain and together with starch and protein forms the composition of most importance for the functionality of the barley grain readily analyzed, at the phenome level.

In the starchy endosperm, starch biosynthesis is characterized by a committed pathway of ADP-glucose pyrophosphorylases (AGPases, EC 2.7.7.27), starch synthases, starch branching enzymes, and debranching enzymes overlaid on general hexose and hexose phosphate metabolism (Schulman (1999), Chemistry, Biosynthesis, and Engineering of Starches and Other Carbohydrates, In: Molecular Biotechnology for Plant Food Production, O. Paredes-Lopez, editor, ed.: Technomic Publishing Co., Inc., Lancaster Pa. USA., pp. 493-523; Smith (1999), Curr. Opin. Plant Biol. 2, 223-229). Together, the Schulman, Opsahl-Sorteberg, and Jansson groups have isolated and characterized a large number of genes for these various enzymes (Rudi et al. (2004), Hordeum vulgare gene for AGPase small subunit (GenBank Accession no. AY634681); Doan et al. (1996), Plant Mol. Biol. 31, 877-886; Thorbjørnsen et al. (1996), Biochem J. 313, 149-54; Sun et al. (1998), Plant Physiol. 118, 37-49; Sun et al. (1997), New Phytol. 137, 215-222; Sun et al. (1999), Plant Mol. Biol. 40, 431-443) and for barley in general as represented in the Schulman EST collection at http://www.ncbi.nim.nih.gov/entrez/ and in a local database. AGPase (EC 2.7.7.27) is a main regulator of starch synthesis in plants and glycogen in bacteria. AGPase catalyses the conversion of glucose-1-phosphate to ADP-glucose (ADP-Glc), the substrate of starch polymers (amylose and amylopectin). The enzyme is a heterotetrameric enzyme consisting of two small and two large subunits, and encoded by different genes expressed in different locations (Preiss et al. (1991), Biochem. Soc. Trans. 19, 539-545). The enzyme is largely extra-plastidial (85-95% cytosolic) in cereal endosperm, plastidial in other cereal plant parts and exclusively plastidial in all tissues of non-cereal plants (Beckles et al. (2001), Plant Physiol. 125, 818-27).

The small subunit (SSU) in the absence of the large subunit (LSU) is able to form an active enzyme, and is suggested to be the catalytic subunit. In contrast, the LSU expressed in the absence of the SSU is unable to form an active enzyme (Ballicora et al. (1995), Plant Physiol. 109, 245-251; Doan et al. (1999), Plant Physiol. 121, 965-975) and is indicated to have a primarily regulatory role. However, Cross et al (Cross et al. (2004), Plant Physiol. 135,137-144) showed that both subunits are involved in the allosteric regulation of AGPase.

There are at least three different AGPase SSU transcripts present in barley grains during seed development and starch accumulation encoded by the two genes HvAGPaseS1 (Thorbjørnsen et al., 1996, supra) and HvAGPaseS2 (Johnson et al. (2003), Plant Physiol. 131, 684-96). Cell-fractionation studies showed that most of the AGPase activity in the endosperm is cytosolic (Denyer et al. (1996), Plant Physiol. 112, 779-85; Sikka et al. (2001), Plant Science 161, 461-468; Tetlow et al. (2003), J. Exp. Bot 54, 715-725; Thorbjørnsen et al. (1996), Plant Journal 10, 243-250). This indicates that the endosperm cells harbour the enzyme in the cytosol as well as in the amyloplasts, and the cytosolic localization of AGPase in cereal endosperm has functional significance for partitioning large amounts of carbon into starch when sucrose is plentiful (Beckles et al., 2001, supra).

Grain starch quantity and quality has been altered by mutagenesis. Upregulated allosteric variants were generated using random mutagenesis to alter the wild-type potato AGPase (Greene et al. (1998), PNAS USA 95, 10322-7). The barley AGPase isoform located in the cytosol is insensitive to 3-PGA/Pi regulation and has a relative high activity without 3-PGA and can hence be used to produce starch in high yield in potatoes or other crops (Patent applications WO 91/19806 and WO 94/24292). Expression of glgC from the patatin promoter (pMON16950) in potato results in enhanced starch content (30%) in tubers (Stark et al. (1992), Science 258, 287-292).

### BRIEF SUMMARY OF THE INVENTION

In one embodiment, the present invention provides a method of preparing a seed product comprising the step of subjecting one or more seeds having low starch and high β-glucan to one or more processing steps. In a particular embodiment, said starch is 25-40% of the seed, dry weight. In another embodiment, said β-glucan is 10-25% of the seed, dry weight. In additional related embodiments, said starch comprises 8-35% amylose and the remainder is provided by amylopectin. In particular embodiments, said seeds are subject to grinding, cracking, dehulling, flaking, defatting, roasting, toasting, extraction and/or extrusion. In a particular embodiment, said processing additionally comprises the addition of further ingredients and suitable processing steps to generate palatable products. Such ingredients may include, in particular embodiments, eggs, milk, sugar, salt, yeast and/or fat are added. In one embodiment, said product is edible. In certain embodiments, the product is meal, flour or flakes, which, in certain embodiments, are processed further to produce animal feed, breakfast cereals, snack foods, pasta, bread, pastries, potato-based foods and/or confectionary. In one embodiment, the seed is selected from the list consisting of wheat, barley, rice, sorghum, oats, rye, triticale and maize (Zea mays). In further embodiments, said seeds are from Risø mutants 13, 16, 29, Perga mutants 95, 449 or waxy line w1.

In particular embodiments, said seed is modified relative to the wild-type seed. In one embodiment, an existing endogenous gene is modified or mutated or exogenous nucleic acid material is added. In another embodiment, said seed is derived from plants or plant cells which have been transfected with sense nucleic acid molecules comprising an unmodified, modified or mutant sequence or with an antisense sequences to the wild-type sequence to impair expression of the wild-type sequence. In a particular embodiment, said mutation is in the lys5 locus in chromosome 6 or in chromosome 7. In a particular embodiment, the sequence which is modified or mutated is brittle-1 (Accession number AY033629) α-glucosidase (Accession No. AAF76254.1) or 3-glucanase (Accession No. AAL73976.1). In a further embodiment, one or more of the genes encoding at least one of the AGPase components is modified, preferably mutated, such that the seed exhibits lower levels of AGPase or lower levels of AGPase activity relative to wild-type. In another embodiment, the sequence which is modified or mutated is the AGPaseS1 gene, preferably from barley, wheat, maize or rice. In yet another related embodiment, the gene which is modified or mutated comprises a sequence specifically recited herein or a portion thereof, or a sequence which hybridizes to said sequence or portion thereof under non-stringent binding conditions of 6×SSC/50% formamide at room temperature and washing under conditions of high stringency, or a sequence which exhibits at least 80% sequence identity to said sequence or portion thereof, or a sequence complementary to any of the aforesaid sequences.

In a further embodiment, the present invention includes a seed product obtainable by a method of the present invention.

In another embodiment, the present invention provides a method of preparing a seed having low starch and high β-glucan comprising at least the steps of: a) inserting an exogenous nucleic acid sequence as defined in any one of claims 14 to 20 into one or more plant cells; and b) obtaining or propagating a seed therefrom.

In a related embodiment, the present invention provides a method of obtaining a seed having low starch and high β-glucan comprising at least the steps of: a) preparing a modified or mutated seed, preferably by random mutagenesis; b) assessing the level of starch and β-glucan in said seed; and c) selecting a seed having low starch and high β-glucan.

The present invention further provides a method of screening for seeds having low starch and high β-glucan, comprising at least the steps of: a) determining one or more phenotypic characteristics of one or more positive seed standards with low starch and high β-glucan; b) determining said one or more phenotypic characteristics of one or more negative seed standards; c) generating a fingerprint representation of the results of said phenotypic characteristics determined in steps a) and b), wherein said fingerprints for said positive and negative seed standards are separable; d) determining said one or more phenotypic characteristics of a test seed and generating a fingerprint representation using the method of step c); and e) comparing the fingerprint generated in step d) with the fingerprints generated in step c), wherein correlation of the fingerprint to the positive or negative seed standard is indicative of the presence or absence of low starch and high β-glucan, respectively.

In one embodiment, the method of screening for seeds having low starch and high β-glucan, comprises at least the steps of: a) performing Near Infrared Reflection spectroscopy on one or more positive seed standards with low starch and high β-glucan to generate spectral traces for said standards; b) performing Near Infrared Reflection spectroscopy on one or more negative seed standards to generate spectral traces for said standards; c) performing Near Infrared Reflection spectroscopy on a test seed to generate a spectral trace for said test seed, and d) comparing the spectral trace generated in step c) with the spectral traces generated in steps a) and b), wherein correlation of the trace to the positive or negative seed standards is indicative of the presence or absence of low starch and high β-glucan, respectively. In a particular embodiment, said spectroscopy is performed at one or more of the following wavelengths: 1100-1400 nm, 1400-1800 nm, 1800-2500 nm, 1890-1920 nm, and/or 2260-2380 nm. In particular embodiments, methods of the present invention include optionally propagating the seed having low starch and high β-glucan for one or more generations.

The present invention further includes a seed identified by a screening method of the present invention. In one embodiment, said seed has been produced by adding exogenous nucleic acid material to said seed or plant or plant cells used to generate said seed.

In yet a further related embodiment, the present invention includes a method of isolating β-glucan comprising the step of isolating β-glucan from a seed, wherein said seed has low starch and high β-glucan.

In another embodiment, the present invention provides a β-glucan preparation obtained by a method of the present invention.

In a further embodiment, the present invention includes a pharmaceutical composition comprising β-glucan obtained by a method of the present invention and a pharmaceutically acceptable diluent, carrier or excipient.

### BRIEF DESCRIPTION OF THE SEVERAL VIEWS OF THE DRAWING(S)

FIG. 1 shows A) NIR (MSC) spectra 400-2500 nm of the 54 barley lines growth in the greenhouse (Group 1), B) PCA scoreplot (PC1:2) of whole NIR spectra in A, Nb=Bomi, Nm=Minerva, for mutant identification see Example, C) Mean spectra from enlarged area 2260-2380 nm (marked with a square in A) of the four genotype clusters (normal, lys3, lys5 and lys3a5g) revealed from the PCA in B, D) comparison of spectra (2260-2380 nm) from lys5f, lys5g, lys3a, lys3b, lys3c, lys3m and the mother lines Bomi and Minerva (lys3m);

FIG. 2 shows A) variation of the amide/protein ratio (ordinate) with β-glucan content in lys5f, lys5g, lys3a, lys3b, lys3c, lys3m, lys3a5g and normal (N), and B) variation of starch content (ordinate) with β-glucan content (lines as in A)); and

FIG. 3 shows A) PCA (PC1:2) of NIR (MSC) spectra from the 54 barely samples (Group 1) grown in the greenhouse and 9 samples of six different mutants in the test set (Group 2) discussed in the example, B) comparison of spectra (2260-2380 nm) from the mutants grown in the green house lys5f, mutant 449 and mutant 95 grown in the field* and in outdoor pots**, C) comparison of spectra (2260-2380 nm) from the mutant lys5g, mutant 16 and normal barley, D) comparison of spectra (2260-2380 nm) from the mutant w1, w2, lys5f, lys5g and Bomi.

### DETAILED DESCRIPTION OF THE INVENTION

The aim of the present invention is to provide low starch seeds, preferably grain, but with high β-glucan. The inventors have surprisingly now identified seeds satisfying these criteria and identified the genetic modifications required to produce such seeds.

Thus, in a first aspect the invention provides seeds, preferably grain, having low starch and high β-glucan levels.

When referred to herein the “seed” denotes a viable entity capable of propagation under the appropriate conditions and produced as a discrete entity from its parent plant. As referred to herein, a “grain” refers to the seed of a plant, suitable for and intended for use in feed or a food product. Thus, the grain may be a seed or may be in a non-viable state, e.g., dried. Especially preferably such plants are cereals, i.e., strains of grasses cultivated for their grain. Such grain include barley and non-barley grain such as wheat, rice, sorghum, oats, rye, triticale and maize (Zea mays). Preferably however said grain is barley.

As referred to herein “starch” refers to polymers consisting of amylose and amylopectin and may be measured by appropriate tests such as described by Munck et al. (2001, Analytica Chimica Acta 446, 171-186).

Preferably the seeds of the invention and for use as described herein contain moderate amounts of amylose. Thus in a preferred aspect, the invention provides seeds having low starch (e.g., less than 50%, dry weight, as described hereinafter) and high β-glucan levels, wherein said starch comprises more than 20%, e.g., more than 15, 10 or 8% amylose, e.g., from 8 to 40% amylose and the remainder of the starch is provided by amylopectin. Especially preferably the starch comprises from 8 to 30%, 10 to 40% or 15 to 35%, especially preferably 10 to 35% or 20 to 30% amylose with the remainder of the starch proved by amylopectin. Values as quoted are when measured on dried seed (dry weight) and harvested from plants grown in a greenhouse, although the ratios described are similarly applicable to plants grown elsewhere, e.g., field grown. Thus, preferably grain of the invention do not extend to waxy grain which have high amylopectin and low amylose.

β-glucan refers to a polymer of glucose molecules comprising 1,3-β linkages (optionally additionally comprising other linkages, e.g., 1, 6 or 1,4 linkages and may be linear or branched in form) and may be measured by use of the enzymatic (1-3, 1-4)-β-glucan kit (Megazyme Intl. Ireland Ltd, Wicklow, Ireland) or the fluorometric BG analysis with Calcofluor as described by Munck et al. (1989, Monatsschrift für Brauwissenschaft 4, 162-166).

Low starch refers to a reduction of at least 20, preferably 30, 40, 50 or 60% (e.g., 20-60% reduction) relative to wild-type seeds (in the case of mutants) or relative to levels commonly found in the seeds in question (e.g., wild-type). Thus for example on an individual seed basis, the low starch seed may have a starch content of less than 50%, preferably less than 45, 40, 35 or 30% (calculated using the dried seed, e.g., 25-40%, dry weight). This is compared to the approximately 50% starch found in barley seed (e.g., Bomi or Risø). High β-glucan refers to an increase of at least 50%, e.g., at least 100, 200 or 300% (e.g., 50-300% increase) relative to wild-type or normal levels (e.g., as found in Bomi or Risø). Thus levels of for example more than 10, e.g., more than 15 or 20% of the seed, dry weight, e.g., 10-25%, are contemplated. Normal seed, for example barley, contains around 5% β-glucan.

The above absolute values in seeds are based on the values obtained when the seeds are harvested from plants grown in a greenhouse. The relative values compared to wild-type or normal material is applicable regardless of the growing site, e.g., greenhouse or field grown.

“Wild-type” as used herein refers to a normal plant line (or a part thereof, e.g., a seed) which has not been mutated or modified as a result of human intervention. Generally this will be the line (or part thereof used to generate a mutant line to which comparison may be made.

Preferably said seed is modified (e.g., is a mutant), i.e., it represents a genetic variant relative to the wild-type seed. The modified or “mutant” seed may be from an individual, or lineage of individuals possessing a modification or mutation, preferably a heritable modification/mutation.

A “mutation” includes a mutation of the genome by deletion, insertion or modification of one or more bases in said genome. Thus a mutation may comprise insertion, deletion or modification of a relevant portion of a relevant gene. Alternatively the seed may be modified by insertion of an exogenous gene or portion thereof into the genome of the wild-type seed (or plant from which the seed is derived).

In a further alternative, genetic or non-genetic material may be added to said seed or the plant producing said seed, to prevent or impair expression of a target protein, e.g., the use of antisense, RNAi or cosuppression techniques or the addition of a molecule which inhibits expression or the activity of the target protein, e.g., an antagonist of a target enzyme.

Thus for example the wild-type seed (or plant or plant part from which said seed is derived) may be subjected to genetic manipulation, e.g., random mutagenesis, site-directed mutagenesis or transgenic manipulation by the introduction of one or more nucleic acid molecules into the seed or a seed or plant (or plant part) from which that seed is derived. The nucleic acid molecules are preferably incorporated into the wild-type seed's genotype.

The seed (e.g., grain) of the invention may be the seed directly produced by genetic manipulation (of the seed or plant part or plant cells used to propagate a plant which produces the seed) or may be the progeny thereof by self-crossing or crossing with another parent. The seed of the invention thus also extends to progeny thereof, e.g., progeny of the plants generated from the seed as well as the plants of that seed and its progeny and plant parts thereof. Thus, for example, the invention extends to the grain (or seed) described herein or the M1, M2, M3 etc., or F1, F2, F3, etc. populations wherein M1 refers to the progeny of seeds (and resultant plants) that contain the modification/mutation, while the M2 population is the progeny of self-pollinated M1 plants, and so forth and F1 is the progeny resulting from cross pollinating one line with another line and the F2 population is the progeny of the self-pollinated F1 line and so forth.

As mentioned above the modification/mutation is preferably heritable, i.e., the desired trait is inherited in progeny.

Various modifications/mutations may be made to generate a seed having the desired properties.

In a particularly preferred embodiment, one or more of the genes encoding at least one of the AGPase components is modified, preferably mutated, such that the seed exhibits lower levels of AGPase or lower levels of activity relative to wild-type, especially preferably significantly lowered AGPase activity, e.g., less than 50% activity, for example less than 20% e.g., no detectable activity. Especially preferably this activity is lowered to such levels in the endosperm in seed.

Conveniently this may be achieved by incapacitating or removing one or more of the subunits making up AGPase, preferably the SSU. Lowered AGPase activity is conveniently achieved by preventing or lowering expression of one or more of the AGPase components. AGPase activity may be assessed as described in Smith (1990, Methods Plant Biochem. 3, 93-101, assay 2b).

Preferably the AGPase activity of seeds is less than 4 μmol.min⁻¹ g⁻¹ fresh weight, preferably less than 3 or 2 μmol.min⁻¹ g⁻¹, compared to approximately 5μmol.min⁻¹ g⁻¹ for wild-type barley.

As mentioned above, in barley at least three different AGPase SSU transcripts exist. In seed, to reduce AGPase levels or activity in accordance with the present invention, cytosolic AGPase levels or activity in the endosperm is preferably reduced. Conveniently the gene encoding the SSU subunit may be modified or mutated. In barley the SSU1 encoding gene, namely Hv.AGPaseS1 is preferably modified or mutated. Conveniently the mutation may comprise deletion of a relevant portion of the encoding gene or mutation thereof. Risø16, a Risø mutant described in the Examples, contains a large deletion in the coding region of Hv.AGPaseS1 and consequently lacks cytosolic AGPase and thus lacks AGPase activity in the endosperm.

This mutant produces only 44% of the starch content of normal seeds (Tester et al. (1993), J. Cereal Sci. 17, 1-9). This has also been shown in quantitative real-time PCR experiments revealing its importance to starch accumulation. Preliminary microarray hybridisations (Timothy Close, Affymetrix Barley GeneChip, Riverside, Calif.) on barley wild-type and the Risø16 mutant (seed 18 DAP) confirmed that both the HvAGPaseS1 transcripts are absent in Risø16 but expressed in wild-type (data analyzed using the GeneSpring software, data not shown). The HvAGPaseS2 and LSU transcripts are present in both wild-type and Risø16, which confirms that only the HvAGPaseS1 gene is affected.

Known mutant seeds (e.g., the mutant grains described herein) having the properties described above do not fall within the claimed seeds. They may however be used in methods and seed products described hereinafter.

Similar modifications or mutations may be made in related genes or alternative modifications or mutations having a similar effect may be made in such genes.

Thus in a further preferred aspect the present invention provides seeds (e.g., grain) having low starch and high β-glucan levels, wherein said seeds are modified (preferably mutated) such that the seeds exhibit lower levels of AGPase or lower levels of activity of AGPase, wherein preferably a modification or mutation is made in the gene encoding the small subunit of AGPase, preferably cytosolic AGPase, wherein said gene which is modified or mutated comprises the sequence:

TGTTTTTTGTGTGTGAATAAACTTGTTGCCAATAAAGCGAAGAGCATATGTAGT
ACGCCAAAAACTTTACAGCTTGTCACATGCGAACTAATTTCGTCGCACATGGA
TATTCATGTGCTCTTTTTTGTACGTGCATATACTTCGTTCGCCTATAAATAAAA
GAAGAGTTTCCTTATGACTTCAAAAGTGAACTCACACATCACTCAATATCTATA
TCCTTCCATTTTATATCCCTCGGTGATGGATGTACCTTTGGCATCTAAAGTTCC
CTTGCCCTCCCCTTCCAAGCATGAACAATGCAACGTTTATAGTCATAAGAGCT
CATCGAAGCATGCAGATCTCAATCCCCATGCTATTGATGTAAGTGGTGCTATC
TTAACTATGATTTTCGTTTTCTGTTCCATCTTTGAGTATCATATGGAGTAATATT
ATTTTTAGGAAGTCTTAGGAAAGGCTTCTTTGGGGCAGCTTCAAGCATAATTA
AAAGACACTCCAGAGCCACATCATCACATGCATGCATATACAACACAACACAA
CACATGAAGTGAGCGATATCTTTTTAGTTTTTCGAACTCCAATTTTTTTCTCTTC
AATAAAAAATACGATTAAAAATCCATTCTCATCATTAAATCTGCTTCCACCAGA
TTTTTAAAACTACATCGCATATTGATAAGTTTCAACGATATTTTTTGGGCCAAA
AGTTATCATGATGCTTACCCTCAAGTTAACATAGTGTTTACACTAAAGTTGTCA
TGATATGTTTTATATATAATTTTCTCAAGATTTTAAAGCTACCATGGAACATGAC
AAATTTAAGTAATTCACCATGACAATTTTAATTTATGGTTCACGTCAAAATTAAG
TCATTGACTATGCATTTTTAAAGTAATTTAACGACATATTTTGTTTTAGTTATGA
ACCGTGCCAAAAATATTTCATGGATCATGGCAATTTTTCATAATTCACCATGAT
AAGTTTTAGTTTCTTATTTTTTTATAACATGACAAAATTACTTTAAATGTAGAAG
AAAAAATTGTTAAAATATATCATGATAACTTTAGTGTAAAAACAATTTATGTGCA
ACAAACATGACAGCTTTTAACCCCAAAAAGCCATTGAAACATATGAAATCTAGT
TTCAGAAATCTCGTTGTGACGAATTTAATGATGAAAATATGTTTTCAATCGGAT
TTTTAATTTAAAAGATAAATCATTTTAAAAGCTAAATTTTTAAAAAAAATATTGCC
ATCATTAGTTTCATCATTTATGCATACATGCGATGATATAGTGTGATGTGTGCG
GATGAATTCGTTCGGTCGATGATCTCCCCGATTGAATGTGTGAACGCTATCAG
AGTCTTTATCTTCCACTGTTCTATCTTATATATTACGTTTTTTTTAAAATTATTGT
CTTAAATTTATCTAGCTACAAATATATCTAACCTTAAAACACGATTAGATACATC
CGTTAGATAAATCCATGATAATTTTTTTAAGACGGAGAGTATGTGCTATGCCTG
TCAAGCAGAAAAATCTGAAAAGACAATTAAGAGAGAAAGAAGTTTACCATTGA
TATTCCAAAATCATCGTGGCTATGTACTCCTCAAGGAGGTCATTCCAAAAGTG
CCGCTGCTCACGATCTCTCTCACTCCCACGCAGCTCTCTCTAAAAGAAAAATG
GCAAAAAACTGAAAATGGAAAGATCTTACGAAAAGATTAGTTAAATTTACTCAG
CCACACTGCACCACTCGGTGTCAGGCGTATCTCTCTCCCTTACCCCTCGTGA
TCTCTCGCCACGGGAGCCCCGTGACTCGAGCTCGTCATCCACCTCAATGGC
GATGGCCGCGGCCGCCTCCCCTTCCAAGATCCTGATCCCTCCGCACCGAGC
CTCCGCCGTGACCGCTGCCGCGTCCACCTCCTGCGACTCCCTCCGCCTCCT
CTGCGCGCCACGAGGACGGCCAGGCCCGCGCGGGTTGGTCGCGCGTCCGG
TTCCGCGGCGGCCCTTCTTCTTCTCCCCACGTGCCGTGTCAGACTCCAAGAG
CTCCCAGACTTGTCTCGACCCCGACGCAAGCACGGTACGCCGCCTCGCCTA
GCCAAATGCGGCGCTTCTTGGCCGCCTAGTCTTGTCTCGCTGCCCTGATCCG
TTGCGTCCGTATCTTTCCGGATGAGAATTTGACACATGCGGGAAGTTATTGCC
TCGGTAATTTAGATGCGCAAATGTGGTTCGCGTCTTGTGTTCTCATGTGGACA
TTTCTTAGAGATGATTAACAAAAAATACTACTCTACTTTGCTACTAGCGCATAGC
ATGAACTTTTGACTAATCATGTGGACATTTTCATTTGCTCCAATTATTTATTTGT
ACTAGATTTCAGTAAATGTGGCAACTGTGGGCGTTTGTTGTACAGTCCTACTT
AATCAATTGGTGTGGGTCTGACACGTGTGAGCCCCATAAGAAATTTATAAAGA
AGATGGCATGATCCATGACATGTGGCTCTTAAAGACCACTGGGCAATCAGAT
CAAGTTCTTGGATTTTTATTTGTAACTTATTCAGTTTTCTTCTTTGAGTTTTGCT
TCAGTACACCCTTTATAAAAACATTACGATTTTGGATGTGGTGGACATTAAAAC
TTACCCTTTCATTTTAATTAAAAGGGTAGTAGAGTCTATTCCACGTGGTGCAAA
TCAATGGTGGTTGCCTCTCTTCCTCACAAACCGTTCTGGCATCAACACAACTA
AACAAAGTAATACAACCAGGCGAGTTTTAGGCGAGATAATAGATTGGGGTCTT
CTGTGCTGGTGAAGACTCTACGTGATGTGAAAAAGTTATACACAACAGGAATA
ACTTGGATCACATCTCAGCTGCAATGCTGATTGAGACATCTGACGTCCAATTA
AACCCATATTCGGAAGAAAAAACTAAACATAAGTTCCAGCTTGATTGATAAATA
AAAGGTCAGAACTATTCACCAGCCAGATGCCCAGTATCTACAAAAGATTGGTA
GACATTGTAGCTTCAGTTTATTGGATAAACTTGTTGCCCCATGTCACATTCATT
CCATGATCTCTTTTGGTGATAAACAAGCTGAACTCAGTGCCAACCGTCTGGAA
CACTTGTTTCTTCGTTCTTTGTTTGATTTACTTTGCAATAGGCAATTGATGAGTT
TCTGCTGTTTGTGCAGAGTGTTCTCGGTATCATTCTTGGAGGTGGTGCAGGG
ACTAGATTGTATCCCCTGACGAAGAAGCGTGCAAAGCCTGCAGTGCCATTGG
GTGCCAACTACAGGCTTATTGATATTCCTGTCAGTAATTGTCTGAACAGCAAC
ATATCAAAGATCTATGTGCTTACACAGTTCAACTCAGCTTCTCTTAATCGTCAT
CTCTCACGAGCCTATGGGAGCAACATTGGAGGTTACAMGAATGAAGGATTTG
TTGAAGTCCTTGCTGCACAGCAGAGCCGAGATAACCCTGACTGGTTCCAGGT
ATCTCATTCATTGTTATTTAAGTGTTTTTGTTTAATGTGAAATGCGAGATTCATC
TACTGATGAACATCATAATTTGTCTCATGTTAGCATTTAGAAGAAGGCAAAATC
TATAATTCCTTCATAAGTACTCGTGATTGTATCATTTCACCCTCTGTGGAAATC
CCAGGGCCAGCCTTCCAAGAACCAGAATAGAAAAGAGACAATCTGTTCCAAG
ACGTCATTGATATTCCTTTTTACAGAACCTTGATGTAGATTATAAGAATTATTAT
TTGGATACTGCCCTAATAGTCCTCTATTTATTATTTCCGATTTTCTAAATAATTC
AATTTAATAGCATGCTATCACACCACAGTTTTAAGGTCAAGTAGAGATGCTCA
GAAATTTTCATGAATTGATTTTAACAGTGTTTCTGAATTATACGAATCTGTTTTG
CGTACCAAGATCTGGTCCTGAACAAGTTCACTAGTTGCAAATTTTGAATTAGT
ATACGTGAATGGTCAGTGATGTAACTTTGATTTTGATTCTTATGAGCATTAGCC
AGTCATCATCATTTATAAGTAAACACAGCAGATCAAACTATGTTTCATACTTTC
GTATGTTTGCCGTTATAATAATACTATTCATCATAGCTTCTGCTTTAGATTGCG
AGTGCTATACCACACAGCTACATGCAGTTTCTGCTATTTTATGTCAAATCAGTT
ACCCTACAGCGTTTTTCTAGATAATAAGAACCAAAGTCATGTCCGTGAGGACT
TGAACCTGGGTGGCTGGGCTGTAGATCCACTCCCCTAACAAAGTGAGCTCTG
CTCACTTCTTGATAATCATAAACTACATAAAGTGTTGCTAGGGTCCCATGCAA
GCTTTTGTAGGGTATTCACTTTGTCCTATCATCTTACCTCAGGGTACTGCAGAT
GCTGTAAGGCAGTACTTGTGGCTATTCGAGGAGCATAATGTTATGGAGTATCT
AATTCTTGCTGGAGATCACCTGTACCGAATGGACTATGAAAAGTTTATTCAGG
CACACAGAGAAACGGATGCTGATATTACTGTTGCTGCCTTGCCCATGGATGA
GGAACGTGCAACTGCATTTGGCCTTATGAAAATCGATGAAGAAGGGAGGATA
ATTGAATTCGCAGAGAAACCATAAAGGAGAACAGTTGAAAGCTATGATGGTACA
CTGACACTGTGCCTTTCTAACTAATTTCAGATATACAGTTGTGAACCATCATTC
ATTACACCACAAAATCTCTTCTGTTGAATGCATTTACACCATGTTGCTACCTGT
TTTGGTCTTGTAATGGTACACTGGCGCTGTGCCTTTCTAACTAATTTCAGATAT
ACAGTTGTGAACCATCATTTATTACACCAAAAATCTCTTGTGTTGAATGCATTT
ACACCATGTTGCTGCCTGTTTTGATCTTGTAGGTTGATACGACCATACTTGGC
CTTGAAGATGCGAGGGCAAAGGAAATGCCTTATATTGCTAGCATGGGTATCTA
TGTTATTAGCAAACATGTGATGCTTCAGCTTCTCCGTGAGCAATTTCCTGGAG
CTAATGACTTCGGAAGTGAAGTTATTCCTGGTGCAACTAGCACTGGCATGAG
GGTAGGCAAAGCTCATTGAGTTAGTAGTTTTTTTTCGCTGCTTCTGCTTTTATG
ATTTGAATCATTTTAGCCTCAGAGAAACTGTCAAGTCATATGTTTATCGTTCGG
AAAGGGATACAATAGGTTATTGGATATGCACTTTGTAGAAACGGGAGGGGGA
GAGGACTACCTCCAGATGGGTCATGGGTGTTGTGGATGTGTGGCGGCTGGC
TCACTCGGGAGGACTGGAAACACCTCCTTCTAGGTCATGTCAAGGGCTAGGC
CTTCCGGGCTTAAGTGAGATGGGCCATACAGCCCATACCGGTTCAACACTCC
CCCTCAAGATGGGTGGTAGATATCTAGCATTTCGATCTTGTAACATGCCAAGT
TACAGTCCTTTGTTCCCAGTCCCTTTGTCAAGCAATCTGCGAACTGTTGCCAC
AAGTTTCAGCACACACCCGCGTGCAATCCACTTGCACCCTGACTCGATGCAA
GCCGCATCATAGAGTTCCAAAGGATCACAATTATCCTGCTCCGCCCACAAAG
CCTGCAACTCTGCCACATATGCCATCACGGACATGTCGTCACCTTGGCGCAA
CCGACTGATCTTCCCCTCAATCTGAGCGATTAGCATGAAATTACCCTTGCCTG
AGTATTGGGTGGATAGGGTCTTCCATATCTCGGAGTGGATAGTCCCTCCACA
GAGCGTCCAATGGAGGGCACCACTGAGTTCAACAACCAGCCAACGAGCACG
GAGCTTATGACCTTCCACCTCTTTCCCTCCGCGGTATTCCTGTCTCCTGGTTC
ATCAATCGTATCCAGTAAGTGCCCATCAAGTTCCTTCTGTTCTACGGTCAGCA
ATGCCCTCCTGGACCAACTCTAGTAATTTGTGGCCCCCTCCAGCTTCATGTCC
AGGGGTGACATTTCGAGCTTCTGAGCCACTTCCTGTCGAGGAACGATGGCCC
CAGAGTTGGACTCTGCGAGGATCTTAGCGAGCTTCTCGAAAGCCTCAGCAAG
CGCATTTGGTTCAGCCATCTCCGATCCGGTAATCAGCAACAGCAGCCCTCAG
CAGCACAGCCCTACAGCTGCACGGTTGGTCCCTTTACGCCCCCTGGCTAGCA
GCAGCACCCAGCAGCTCCAAGTGCAAGCAGCAACCACAGCCTCTTCCTCCAG
CAGCAGTTCTCTTCCTTCTTCCTCTGCAAACAGCAGCAGTTCTCTTCCTCCTT
CCTCTGCAAACAGCAGCAGTTCCAGAGGATCTTGGCCTCCAACAGCAGCCAA
CCCCCGCAGCAGTTCTCCAGCTCCCGGTCCCCTGCAGACACACGCACACAC
AGCAGCACAACAAGGAGCTGCCCTTCTCCACTATCCTTCTCCTCTTCTCCTCC
CGCAGCACCACAGCCCACCACGCTGAGGAGCAAGCAGCAGCCACAGCACAA
CCCACTCTCGGGGAGACCAAGCCGCTCCTCAGCTTCCTCCTCCTGCCGTGCA
GCACAGCAGCTCCACCTCTCCTTCTTCCTCTGCCGCGCAGCACAGCAGGTCC
ACCTCTCCTTCCTCTCCAACGAGCCGCACCTCCTGCGCAGCCACACCGTGCC
TCCCAGATCCGCCGCTGCCTCACCGGACGCCGCCCAAATCCGCCGCCACCT
CGCTGGACGTCGCCGCCGTCTTCTCAGGCCCCGCCGTCGCTGGCCTCTGAT
ACCATGTAGAAACGGGAGGGGGAGAGAGGACTACCTCCAGATGGGTGATGGGT
GTTGTGGATGTGTGGCGGCTGGCTCAGTCGGGAGGACTGGGAACACCTCCT
TCTAGGCCATCTCAAGGGCTGGGCCTTGTGGGCTTAAGTGAGATGGGCCATA
CAGCCCATACCAGTTCAACACACTTCCATTGGCATTCATAGTTGTGATATGTG
CTTCTTAAGAGTTTTGTTATTGTTGCCGACAGGTACAAGCATACCTATACGAC
GGTTACTGGGAAGATATTGGTACAATTGAGGCATTCTATAATGCAAATTTGGG
AATTACCAAAAAACCAATACCTGATTTCAGGTGCGCTTTCATTTTTTGCCTTGT
TGTGGACAAATATTATGAAATTGCATGCATGTAAAGTGTTAGAATTGTCCCCTA
TTGATTTAATGTATACGTTCAATTTGAATTCAGTTTGTATGACCGTTCTGCTCC
CATTTACACACAACCTCGACACTTGCCTCCTTCAAAGGTTCTTGATGCTGATG
TGACAGACAGTGTAATTGGTGAAGGATGTGTTATTAAAGTAAGTAGCCTTTTT
CAGTTGGCTCTCGGTATGCTAACCCTTCTTCAGGTGTTCCATTTCGTGCTAAC
AAACCTTAAGCTTTTAAAGACATATTTCAAAACCATCTATACTTCTTTATGGGC
TGTGATTGTTATATCTTCTCTCAAGTGATTTTTGATGCTGTGTGTTATAAAGAC
TTCTAAGTTACATTTGCCTTTCTTTGGTCTCCAGGTAGAACTGCAAGATACACC
ATTCAGTAGTTGGACTCCGTTCCTGCATATCTGAAGGTGCAATAATAGAGGAC
ACGTTGCTAATGGGTGCGGACTACTATGAGGTAAAATCAGACAGGTGTAATAT
GCTTCTGCCAAAGTGATGTACTCACCCCTTCTTTTATTGTTCAACAGACTGAA
GCTGATAAGAAACTCCTTGCTGAAAAAGGTGGCATTCCCATTGGTATTGGAAA
GAATTCACACATCAAAAGAGCAATCATTGACAAGAATGCTCGTATTGGAGATA
ACGTGATGGTATGCCATATTGATATACTTATGCTTAAACATCTATTGGTTTCTC
TTTTTCTTTTCCACTGTGGTAGGAACCGCTAAGGTTCTACCGGGTCTAGGGCG
GAGGTTGTAGGGGATGAAGCGGAGTTGGCGAGGGCTGTCTCGCGGCGGCC
GGCGGCGGGACGCCGTTGCGCGCGAGAGGGAGGCGGCGGCGGTGGAAGG
CGGCAGCGCAGCTAGGGTTCCGGCTCCTCTGGGAGCCGGGCAATAGAGTTA
TGACTATATTGCTTAATTCCCAAAAGAGTTGTTTACATTGGTTTATATAATCTC
GATAACTTGGACTCTAAGATCACTAAGATAACTTGGACTCTAAGATAACTTGG
ACACTAAGATAACTAAGATAACATGGGCTAAGCCCGTAACTAATCCTGCCCAT
TGGGCCTGGTCCGTTGGTTCGTAGTACCGGTCATAACACACTGCAACATTGT
CATGCTGAATATGTAACTTGAACATAACTTTTCTCACGGTAATGTCCAAAATGT
AACCATTATATAACAAGCTTTAGGTCTTGTCGGGTTCATAGGAAAAGTGAGAA
AAATGTAGGAAGAGGATATTTTCTTTTAGCGTCACTGTTCATTCGTTTTGTTCA
AAGGAGAAGTGGAGGAAAGTTCCTTTGATCACACTTCGAAAGGAAAATCGCA
GGAATTTTATAATCTACTTGACTTCCGTCTTAGTTATCCTTCTTCATGTGCTTTG
ACTTTGATTTGACTGTCATTAGCGGATGGTTAAGACATGCTGATAATGTCAAG
GGAGGTCGGGGTAGACCAAACTTGACATGGGAGGAGTCTCTAAAGAGTGAC
CTGAAGAACTGGAATATCACCAAAGATTTAGCCATGGAGAGGGGTGTGTGGA
AGTTAGTTATTCACTGCCAGAACCATGACTTGGTTTTGATATCGGATGGATTT
CAACTCTAGCCTACCCCAACTTGTTTGGGACTGAAAGGCTTTGCTGTTGTTGT
CCTATGTTCTATTCCTTAGAAGCAGACTTATATTAGGGTGAAAACTTGTTTTGC
ACTTCCATTGCTACCCTCTTTTTTGTCATTTTCTTTCTATTTCTATGTTATCATAA
TCCTGTGAACCAAACATATCCTATATTGTATATCCATTTCCTTGAACATGATAT
CACGCACTGTGCGTTGTTTTTGGTAGTGATCTGGACTCATTGGTATATTGTAG
ATAATCAATGTTGACAATGTTCAAGAAGCGGCGAGGGAGACAGATGGATACT
TCATCAAAAGTGGCATCGTAACTGTGATCAAGGATGCTTTACTCCCTAGTGGA
ACAGTCATATGAAGTAAGTTGTCTCGTCGTACACACCTCGGTGTCTGCAATCA
GTTATGTTTTATTTTAGAAACTATGAACATGTTGTAAACCAAAAATGATGCAAA
TGCAGCAATACAGTTGGTACATGCAAACCATGCACTGGTATCCTATACATTCA
ATTTGAGATTTTAGCACTCTTCTTGTAAGTAGTTGACTCTGTTTGGGTTGCCCT
GCAGGCAGATGTGAAATGTATGCCAAAAGACAGGGCTACTTGCGTCAGTCTG
GAATCAACCAACAAGGCCGCGAAGAGATCATAAAATAAAAAGGAGTGCCATG
CGAGTCACTTCTACACCCTTTTCCCCCCTTGATGTATTAGGAACTGTGATGTA
CAAGCAACTGTGATGCACTTACGCGAAGTGCCCCTGGATTCAGCTTTCTCTTT
GCTTGTAACTGGTTTCCAGCAGACCATGCTATTTGTTGTATGGTTCGTGCAAA
ACCTTGCGATGCTTTATATATGCTTTATATATAAAAGAAGATGAATCCCGCGCG
TTGCTGCGG

or a portion thereof,
or a sequence which hybridizes to said sequence or portion thereof under non-stringent binding conditions of 6×SSC/50% formamide at room temperature and washing under conditions of high stringency, e.g., 2×SSC, 65° C., where SSC=0.15M NaCl, 0.015M sodium citrate, pH 7.2,
or a sequence which exhibits at least 80%, preferably 90 or 95%. e.g., at least 98% sequence identity to said sequence or portion thereof (as determined by, e.g., FASTA Search using GCG packages, with default values and a variable pamfactor, and gap creation penalty set at 12.0 and gap extension penalty set at 4.0 with a window of 6 nucleotides),
or a sequence complementary to any of the aforesaid sequences.

Preferably such related sequences, but for the modification or mutation, would encode a functional AGPase SSU, e.g., are naturally occurring in cereal plants. The above disclosed sequence provides the full length wild-type HvAGPaseS1 barley gene including introns, from exon 1a to the end of the open reading frame. This nucleic acid molecule and its modified variants/mutants as described above, form further aspects of the invention.

“Portions” as referred to above, preferably comprise at least 30% of the provided wild-type sequence, e.g., at least 50, 70 or 90% of the sequence, e.g., comprise 4000 or more bases.

Preferably, the sequence which is modified or mutated is the naturally occurring sequence found in a seed from a naturally occurring seed, e.g., the above barley sequence or the corresponding sequence in wheat (Ta.AGPaseS1, Embl. Accession No. AF536819, Burton et al. (2002), Plant Physiol. 130, 1464-1475), maize (Zm.AGPaseS1, Embl. Accession No. AF334959, Hannah et al. (2001), Plant Physiol. 127, 173-183) or rice (Os.AGPaseS1, Embl. Accession No. J04960, Anderson et al. (1989), J. Biol. Chem. 264, 12238-12242).

Mutations which are contemplated include deletion or modification of one or more relevant portions of said sequence, e.g., one or more regions of the exons in said sequence. The sequence provided above encodes a protein as described in Thorbjørnsen et al. ((1996), Biochem. J. 313, 149-154), in which at least some of the coding regions are disclosed. Preferably mutation or modification of the above described sequence is performed in these coding regions and/or coding regions of the Hv.APGaseS1 gene disclosed herein to maximize the effect on the product.

Appropriate mutants/modifications may be determined by testing the level of AGPase activity in cells (e.g., the mutated cells, or cells generated therefrom) containing the mutant/modified sequence by the test for activity described above.

Modified/mutated seeds may be generated by mutation or modification of an existing endogenous gene (e.g., by random mutagenesis or site-directed mutagenesis) or by the addition of exogenous nucleic acid material. In the case of the latter, an appropriately mutated or modified sequence may be introduced into plant cells by any appropriate means. Suitable transformation or transfection techniques are well described in the literature. The nucleic acid molecules may be operatively linked to an expression control sequence, or a recombinant DNA cloning vehicle or vector containing such a recombinant DNA molecule. In particular, appropriate nucleic acid molecules may be introduced into vectors for appropriate expression in the cells.

Appropriate expression vectors include appropriate control sequences such as for example translational (e.g., start and stop codons, ribosomal binding sites) and transcriptional control elements (e.g., promoter-operator regions, termination stop sequences) linked in matching reading frame with the above described nucleic acid molecules. Appropriate vectors include plasmids and viruses. Appropriate promoters include tissue specific promoters such as promoters that limit expression to the endosperm.

The generation of transgenic plant cells and the propagation of plants for the generation of transgenic seeds may be performed by well known techniques in the art. For example, vectors may be used for transformation by direct DNA uptake methods (see e.g., Christensen and Quail (1996), Trangen. Res. 5, 213-218, e.g., pAHC25) or with an appropriate carrier system. Preferably transformation is achieved using Agrobacterium carrying vectors to insert nucleic acid material of interest (e.g., pGreen or pCAMBIA vectors, e.g., pGreenII0179, Hellens et al. (2000), Plant Mol. Biol. 42, 819-832). Whilst plant cells may be transfected with sense nucleic acid molecules comprising a modified or mutant sequence (e.g., as described above), plant cells may also be transfected with antisense sequences to the wild-type sequence to impair expression of the wild-type sequence. In such modified grain, for example, the sequence described above may be inserted into the genome of the cell in antisense orientation where its transcripts affect the expression of the wild-type sequence leading to lower AGPase production. Preferred mutants of the invention or for use in the invention are mutated in the lys5 locus in chromosome 6 or in chromosome 7.

Other genes which may be modified/mutated include brittle-1 (Accession number AY033629) which may be down-regulated, whereas α-glucosidase (Accession No. AAF76254.1) and 3-glucanase (Accession No. AAL73976.1) may be up- or down-regulated. Up-regulation may be achieved, e.g., by enhancing their expression in wild-type grain. Modification and mutations may be made in these genes as described above for AGPase.

Seeds of the invention may be produced by the specific means described above in which particular genes are inserted or targeted. Thus a further aspect of the invention provides a method of preparing a seed with low starch and high β-glucan levels, comprising at least the steps of:
a) inserting an exogenous nucleic acid sequence as described hereinbefore into one or more plant cells; and
b) obtaining or propagating a seed therefrom.

Preferably insertion of said sequence is achieved by transformation of the cells with an exogenous nucleic acid molecule such that it is stably incorporated into the genome. The nucleic acid sequence may be in the sense or antisense orientation. Preferably however it is in the sense orientation and is mutated or modified and is preferably as described hereinbefore. Preferably the plant cell is a cell from barley, wheat, rice or maize. Whilst seeds may be mutated or modified directly, preferably plant cells are modified or mutated and seeds propagated by appropriate means, e.g., by propagation of a plant and the development of a seed therefrom. Preferably the mutated nucleic acid sequence is a mutated sequence as described hereinbefore, especially preferably the sequence as mutated in Risø16, as described herein.

Other techniques may also be used to generate grain having the desired low starch, high β-glucan properties. Thus for example random mutagenesis may be performed to generate mutants in the grain and the progeny of the grain may then be analysed to establish if they carry the desired characteristics.

Such mutation may be performed by the use of, for example, ethyl methane sulphonate (EMS), γ-irradiation, thermal neutrons, fast neutrons, ethyleneimine (EI) or sodium azide by well-known techniques.

Screening may be performed by analysis of phenotypic or genotypic traits. The methods described below advantageously may be used for high-throughput screening. Thus, chemical analysis of β-glucan and starch levels may be conducted as described hereinbefore. Alternatively, phenotypic analysis may be conducted, for example as described in the Examples in which spectral signatures are established for grain having desired properties and the spectral signature of a test grain is compared to such signatures to determine if it carries the desired characteristics. The data obtained from multiple grain according to the invention may be assessed by statistical analysis using the Principal Component Analysis method and a score plot can be developed which allows the identification of gene-specific patterns. Test grain may then be classified according to their position on the plot and whether they fall within the clusters having the desired properties.

In a further aspect therefore the present invention provides a method of obtaining a seed of the invention comprising at least the steps of:
a) preparing a modified or mutated seed, preferably by random mutagenesis;
b) assessing the level of starch and β-glucan in said seed;
c) selecting a seed having low starch and high β-glucan.

As appropriate the test may be repeated on progeny of the selected grain to ensure heritability of the desired trait. The screening test described below may be used in this method.

The present invention further extends to a method of screening for seeds of the invention, comprising at least the steps of:
a) determining one or more phenotypic characteristics of one or more positive grain standards (i.e., having the desired traits, e.g., Risø16) with low starch and high β-glucan;
b) determining said one or more phenotypic characteristics of one or more negative grain standards (i.e., not having the desired traits, e.g., corresponding wild-type grain);
c) generating a fingerprint representation of the results of said phenotypic characteristics determined in steps a) and b), wherein said fingerprints for said positive and negative grain standards are separable;
d) determining said one or more phenotypic characteristics of a test grain and generating a fingerprint representation using the method of step c);
e) comparing the fingerprint generated in step d) with the fingerprints generated in step c), wherein correlation of the fingerprint to the positive or negative grain standard is indicative of the presence or absence of low starch and high β-glucan, respectively; and
f) optionally propagating the seed having low starch and high β-glucan for one or more generations.

As used herein “determining” requires that phenotypic characteristics are qualitatively or quantitatively assessed, preferably the latter. Thus for example, a numerical value or other mathematical representation may be assigned to that characteristic.

Conveniently, the phenotypic characteristic which is examined is readily determinable, e.g., a spectral trace as determined in the Examples (near infra red spectral trace). Thus for example a spectral trace at a relevant wavelength range may be used to provide a fingerprint representation of one or more phenotypic characteristics which affect the spectra of a sample. Correspondence of that spectral trace with a spectral trace of a sample with a desired trait (e.g., high β-glucan) or a spectral trace of a sample negative for that trait may be used to determine whether the test sample is likely to have the desired trait. In view of the differences observed between positive (high β-glucan) and negative (normal β-glucan) samples in NIR spectroscopy, one or more of the following wavelengths are informative and may be used for analysis: 1100-1400 nm, 1400-1800 nm, 1800-2500 nm, 1890-1920 nm, 2260-2380 nm or a sub-range thereof or where appropriate a single wavelength if a significant difference between samples with negative and positive traits is evident at that wavelength.

Thus in a preferred embodiment the present invention provides a method of screening for seeds of the invention, comprising at least the steps of:
a) performing Near Infrared Reflection spectroscopy on one or more positive grain standards with low starch and high β-glucan, e.g., at a wavelength of 2260-2380 nm, to generate spectral traces for said standards;
b) performing Near Infrared Reflection spectroscopy on one or more negative grain standards, e.g., at a wavelength of 2260-2380 nm, to generate spectral traces for said standards;
c) performing Near Infrared Reflection spectroscopy on a test grain, e.g., at a wavelength of 2260-2380 nm, to generate a spectral trace for said test grain,
d) comparing the spectral trace generated in step c) with the spectral traces generated in steps a) and b), wherein correlation of the trace to the positive or negative grain standards is indicative of the presence or absence of low starch and high β-glucan, respectively; and
e) optionally propagating the seed having low starch and high β-glucan for one or more generations.

The phenotypic characteristic may determine or reflect the level of protein or other entities, such as metabolites and thus may be analysed by techniques currently used in proteomic/metabolomic analysis. A single phenotypic characteristic may reflect multiple phenotypic phenomenons (e.g., levels of one or more proteins), but provide a single measurable characteristic. More than 1 characteristic may be assessed, e.g., 2 or more characteristics.

“Fingerprint generation” refers to developing a representation of the numerical information reflecting the phenotypic characteristic and may be a numerical value e.g., on a plot or a range of values e.g., a curve or array. Reference to a fingerprint infers that such data is unique to the sample under study. One or more standards may be used in the analysis. Preferably however more than 1, e.g., >5, preferably >10 positive and/or negative standards are used.

Fingerprint representations may be prepared by convenient means, e.g., by statistical clustering methods as described herein. Comparison of the fingerprints is conveniently achieved by appropriate means depending on the fingerprint representation, e.g., by visual or mathematical comparison. In the case of clustering methods described herein, comparison is conveniently achieved by analysis of the position of the test grain on the score plot. Separable fingerprints refers to fingerprints which can be distinguished clearly to accepted levels of significance for the positive and negative samples.

“Correlation” refers to non-absolute similarity, e.g., a common pattern indicative of a test sample exhibiting properties of a positive or negative sample within the error range of the method. A positive correlation with the positive samples confirms the presence of the desired properties in the test sample within the ranges represented by the positive samples.

In an alternative method genotypic characteristics may be examined (using known genomics analysis techniques) and a fingerprint similarly generated, e.g., by microarray analysis.

Such methods may be used for screening for grain with desirable properties. The screening method may also be used to monitor or analyse breeding or mutation programs to select progeny which show improved properties, i.e., show a significant shift to the fingerprint representation of the sample with the desired properties. Seeds selected by the above described screening methods form a further aspect of the invention.

The present invention further provides a seed (e.g., grain) product obtainable from a seed of the invention. Preferably said seed, e.g., grain, product is edible. Such products are obtained by processing methods as described hereinafter. Preferably said products maintain the reduced starch and elevated β-glucan content of the starting material. The seed used for the preparation of the products may be from one or more types of mutant/modified seeds or may be from only a single type of mutant/modified seed. Preferably the grain products (including β-glucan as a product) are derived from the mutant grain described herein, e.g., Risø mutants 13, 16, 29, Perga mutants 95, 449 and waxy line w1.

Additional components may be added as necessary, e.g., in various food products. Preferably the method is achieved without the addition of β-glucan from alternative sources and/or the removal of starch and instead the desired properties are achieved by reliance on the elevated and reduced levels of β-glucan and starch, respectively, existing in the starting material.

The present invention also provides a method of preparing a seed (e.g., grain) product comprising the step of subjecting one or more seeds/grains having low starch and high β-glucan to one or more processing steps, particularly, grinding, cracking, dehulling, flaking, defatting, roasting, toasting, extraction and/or extrusion. Especially preferably said processing additionally comprises the addition of further ingredients and suitable processing steps (e.g., baking) to generate palatable products, e.g., the addition of eggs, milk, sugar, salt, yeast and/or fat. The invention further extends to the products thus produced.

Seed products which may be produced are those which may be used in feeds or foods and include minimally processed products such as meal (e.g., simply milled and optionally dehulled seeds), flour, and flakes which may be incorporated into, or processed further to produce, a variety of more highly processed edible products including animal feed, breakfast cereals, snack foods, pasta, bread, pastries, potato-based foods, confectionary and other products using flour or meal of the invention. The products may be obtained by appropriate physical and/or chemical processing methods, such as heat conditioning, flaking and grinding, extrusion, solvent extraction, aqueous soaking and extraction of whole or partial seeds. The seeds may further be processed to concentrate and isolate particular components, e.g., the β-glucan component. The products obtained or the seeds used for their production may be roasted, toasted, defatted, dried and/or dehulled if desired.

Seeds of the invention which produce elevated β-glucan may also be used as a source of that product which has medical and health uses. Thus the present invention provides a method of isolating β-glucan comprising the step of isolating β-glucan from a seed having low starch and high β-glucan. Preferably the β-glucan is obtained as an enriched, semi-purified or purified preparation, e.g., a substantially pure preparation, e.g., comprising less than 20, e.g., less than 10, 5 or 1% contaminants. Conveniently said preparation is made by a method as described by Symons & Brennan (2004), J. Food Sci. 69(4), 257-261, e.g., by the use of amylase to prepare purified fractions, or by the method described by Izydorczyk (2003, J. Cereal Sc. 38, 15-31).

The present invention thus further provides a β-glucan preparation obtainable from seeds as described herein.

Such β-glucan preparations may be used as a health supplement or in known medical treatments which benefit from the administration of β-glucan. The preparations may thus be provided in a pharmaceutically acceptable format, e.g., containing one or more pharmaceutically acceptable carriers, excipients or diluents.

Thus, the present invention also extends to pharmaceutical compositions comprising β-glucan as described hereinbefore and a pharmaceutically acceptable diluent, carrier or excipient. As referred to herein, “pharmaceutical compositions” includes compositions for medical use (i.e., in the treatment of specific condition(s)), as well as compositions for administration for health benefits, i.e., a nutraceutical, functional food or health food or supplement.

“Pharmaceutically acceptable” as referred to herein refers to ingredients that are compatible with other ingredients in the composition as well as physiologically acceptable to the recipient.

Pharmaceutical compositions according to the invention may be formulated in conventional manner using readily available ingredients. Thus, β-glucan may be incorporated, optionally together with other active substances as a combined preparation, with one or more conventional carriers, diluents and/or excipients, to produce conventional galenic preparations such as tablets, pills, powders, lozenges, sachets, cachets, elixirs, suspensions, emulsions, solutions, syrups, aerosols (as a solid or in a liquid medium), ointments, soft and hard gelatin capsules, suppositories, sterile injectable solutions, sterile packaged powders, and the like.

Suitable excipients, carriers or diluents are lactose, dextrose, sucrose, sorbitol, mannitol, starches, gum acacia, calcium phosphate, aglinates, tragacanth, gelatin, calcium silicate, microcrystalline cellulose, polyvinylpyrrolidone, cellulose, water syrup, water, water/ethanol, water/glycol, water/polyethylene, glycol, propylene glycol, methyl cellulose, methylhydroxybenzoates, propyl hydroxybenzoates, talc, magnesium stearate, mineral oil or fatty substances such as hard fat or suitable mixtures thereof. The compositions may additionally include lubricating agents, wetting agents, emulsifying agents, suspending agents, preserving agents, sweetening agents, flavouring agents, adsorption enhancers, e.g., for nasal delivery (bile salts, lecithins, surfactants, fatty acids, chelators) and the like.

### EXAMPLE 1

### Screening and Identification of Low Starch, High β-Glucan Seed

Near Infrared Reflectance (NIR) spectroscopy was tested as a screening method to characterise high lysine mutants from a barley collection by classification through Principal Component Analysis (PCA). Inspecting mean spectra of the samples within each cluster, gene-specific patterns were identified in the area 2270-2360 nm. The characteristic spectral signatures representing the lys5 locus (Risø mutants 13 and 29) were found to be associated with large changes in percentage of starch and (163, 164)-β-glucan (BG). These alleles compensated for a low level of starch (down to 30%) by a high level of BG (up to 15-20%) thus maintaining a constant production of carbohydrates around 50-55% which is within the range of normal barley.

The spectral tool was tested by an independent data set with six mutants with unknown carbohydrate composition. Spectral data from four of these were classified within the high BG lys5 cluster in a PCA. Their high BG and low starch content was verified. It is concluded that genetic diversity such as from gene regulated carbohydrate and storage protein pathways in the endosperm tissue can be discovered directly from the phenotype by chemometric classification of a spectral library, representing the digitised phenome from a barley gene bank.

The barley endosperm is a well conserved imprint of the physical-chemical dynamics of an approximately 35 day developmental process after anthesis which is regulated by specific genes according to a precise timetable set by the genotype, partly independent of environment. Due to self pollination all advanced barley lines are almost homozygotic and mutants have near isogenic backgrounds for precise references. The desiccated seed/endosperm system is ideal for exploration by near infrared spectroscopy because of reduced interference by water peaks. The phenome (Watkins et al. (2001, Am. J. Nutr. 74, 283-286) is here regarded as an interface expressed as patterns of chemical bonds for the expression of specific genes (Munck, 2003, Detecting diversity—a new holistic, exploratory approach bridging the geneotype and phenotype, in: Diversity in Barley (Hordeum vulgare), von Bothmer et al. (eds.). Elsevier Science B.V., Amsterdam, The Netherlands, Chapter 11, 227-245). These are indirectly observed by spectroscopy as chemical-physical fingerprints.

Material and Methods

The barley mutant genes (Doll (1983), Barley Seed Proteins and possibilities for their improvement, in: Gottchalk & Muller (Eds), Seed Proteins, Martinus Nijhoff/Dr. W.Junk Publishers, The Hague, 205-223) investigated here consisted of:

1. Four alleles in the lys3 locus in chromosome 5 (new nomenclature, see Muravenko et al. (1991) Standardization of chromosome analysis of barley, in Munck (ed), Barley Genetics VI, Munksgaard International Publication, Copenhagen, Vol. I, 293-296) with alleles Risø mutants a (1508), b (mutant 18) and c (mutant 19) in a Bomi background and the Carlsberg mutant 1460 (Munck (1992), The case of high lysine barley breeding, in: Shewry (ed.), Barley: Genetics, Molecular Biology and Biotechnology, CAB International Wallingford, U.K. 573-602) in Minerva here called lys3m.

2. Two lys5 alleles in chromosome 6 Risø mutants 5f in Bomi (mutant 13) and 5g in Carlsberg II (mutant 29) as well as the double recessive lys3a5g.

An independent test set included four high lysine mutants and two waxy lines: the Risø mutants lys4d (mutant 8, in chromosome 1) and mutant 16 (in chromosome 7) both induced in Bomi (Doll (1983), supra), the Italian mutants 95 and 449 induced in Perga by Di Fonzo and Stanca (1977, Genetica Agraria 31, 401-409) and two putative waxy (amylopectin) lines 1201 and 841878 of unknown origin previously imported to the Carlsberg collection maintained at the Royal Veterinary and Agricultural University (KVL) called w1 and w2. Some of these mutants are also obtainable from the John Innes Centre, UK.

The mutants and their parent varieties and segregating crosses with normal barley as well as a range of normal barley varieties were grown under different conditions (field, outdoor pots, greenhouse) in different years.

The material was stored in closed containers in the refrigerator after they were equilibrated to the temperature and moisture of our laboratory. Thus two groups of samples were obtained:

Group 1: 54 normal and original mutant lines (lys3, lys5, lys3a5g) grown in a greenhouse in 1998-2000.

Group 2: Nine lines from the test set of six mutants defined above, mainly grown in a greenhouse.

The NIR analysis (on milled flour from ripe seeds) was carried out as described by Munck et al. (2001, supra) together with the chemical analyses of protein, amino acids, amide, nitrogen, and starch. A determination of apparent amylose content in starch from the two waxy lines was made with an iodine spectroscopic method on non-defatted isolated starch (BeMiller (1964), Iodometric Determination of Amylose—Amperometric Titration). Two methods of BG analysis were employed. The fluorimetric BG analysis with Calcofluor (Munck et al., 1989, supra) was used routinely and was checked with an enzymatic method specific for (163, 164)-β-glucan (Anonymous, 1998). Both methods had a linear correlation with minimal offset up to at least 15% BG d.m (dry matter).

Chemometric pattern recognition analysis of spectral data was performed by Principal Component Analysis (PCA) for classification. The Unscrambler software (Camo A/S, Trondheim, Norway) was used according to Martens and Naas (1989), Multivariate Calibration, John Wiley, Chichester. The spectra were subjected to multiplicative signal correction (MSC) according to Geladi et al. (1985), Applied Spectroscopy 39,491-500. MSC was performed in The Unscrambler.

Results and Discussion

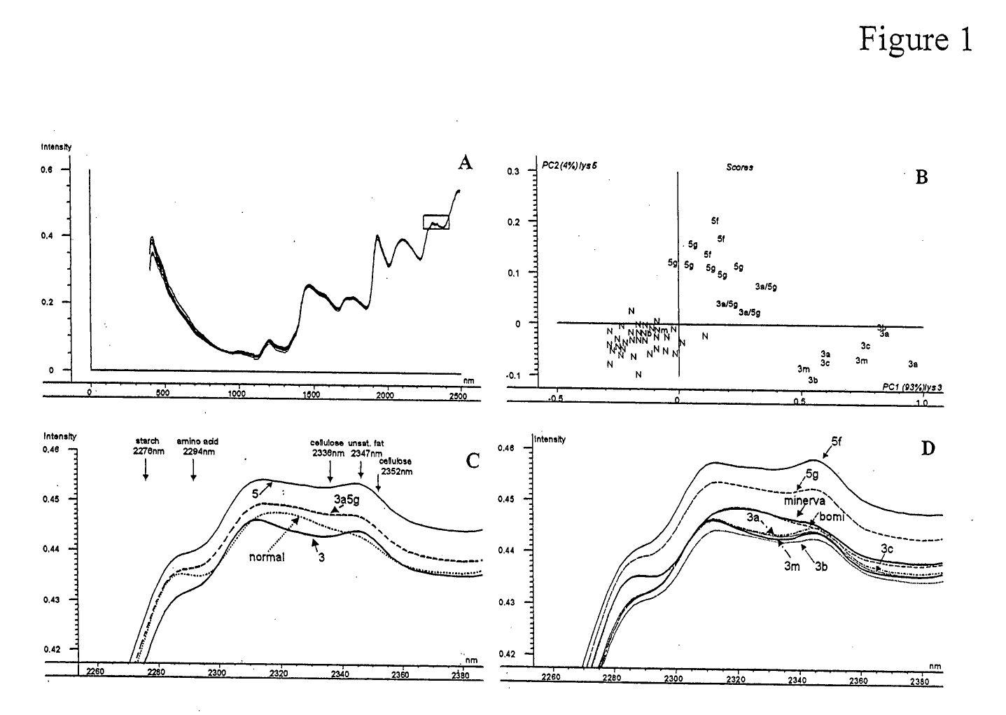

NIR spectra of whole milled flour of 54 barley lines grown in one environment (Group 1, greenhouse) are presented in a multiplicative signal corrected (MSC) form in FIG. 1A. These data were used to develop a PCA score plot (FIG. 1B) between the principal components PC1 (abcissa) and PC2 (ordinate). Three clusters are shown in the PCA plot (FIG. 1B). Empirically observing such interesting patterns, it is natural to try to identify their cause. From the additional information on the samples it was found that the clusters reflect four different genotypes—normal barley N, lys3 (four alleles a, b, c and m) along the PC1 axis and lys5 (two alleles f and g) spanning the PC2 axis and with the double recessive lys3a5g in between.

The evaluation of the PCA score plot in FIG. 1B facilitates a reduction of the 54 spectra to four mean spectra representing the clusters of normal (N), lys3, lys5 and lys3a5g barley. Guided by loadings of the PCA model, the most important wavelengths can be detected and when inspecting these four mean spectra, we can identify an interesting small area in the NIR spectra indicated with a square in FIG. 1A between 2270 and 2380 nm and displayed enlarged in FIG. 1C-D.

The mean lys3 and lys5 spectral signatures in FIG. 1C are distinctly different from each other and from that of normal barley (mean), while lys3a5g (mean) is intermediate between those of lys3 and lys5. In FIG. 1D the same conclusion can be drawn from spectra of individual samples of the four lys3 alleles lys3a, lys3b, lys3c, lys3m and from the two alleles in lys5, lys5f and lys5g. The similar spectral responses for the samples of all the lys3 alleles in Bomi background lys3a, lys3b, lys3c show the same response as the fourth lys3 allele mutant lys3m in Minerva as demonstrated in FIG. 1D. The spectra from lys5f and lys5g have similar form. However, lys5f has a more extreme peak at 2350 nm, which confirms the more extreme position of lys5f, compared to lys5g in the PCA in FIG. 1B. The spectral differences between the barley reference varieties Bomi and Minerva and between most of the other normal lines are small.

As shown above NIR spectroscopy evaluated by PCA is surprisingly effective in differentiating this genetic material grown in a greenhouse. For the best genetic separation, the material should be grown in the same environment (Munck et al. (2001), supra). The spectroscopic signatures indicative for different gene loci and normal barley are clear cut and reproducible. The method is able to differentiate between alleles in the same locus. Thus, lys5f seems to be a more extreme mutant than lys5g. Differences in genetic background within the normal barley category (Bomi and Minerva) and within mutant alleles are less important than the effects of the mutants themselves (Munck et al. (2001), supra; Jacobsen et al. (2004), submitted paper). The NIR spectrum contains repetitive confounded physical-chemical information throughout the NIR spectrum as primary, secondary, tertiary . . . vibration overtones and combination bands from 2500-713 nm emerging from the fundamental vibrations in the Infrared (1R) region 2500-13000 nm.

The NIR detection of the lys3 alleles (FIG. 1B) was not surprising. The lys3a genotype (Munck et al. (2001), supra) is characterised by a low amide/protein N ratio (A/P) of 11.4 compared to its mother variety Bomi (A/P=16.3) due to the low content of hordeins rich in amides. It was then shown that the lys3a gene is likely to be detected by NIR because it mediates low amide content. Information for the amide bond is according to Osborne et al. (1993, Practical NIR spectroscopy with applications in Food and Beverage analysis, Longman Scientific and Technical, Harlow, U.K.) distributed at 20 wavelengths in the NIR area 1430-2180 nm. We also confirmed (Munck et al. (2001), supra) a high correlation between lysine and amide content (r=−0.97).

As discussed below amide detection is only a part of the definition of the lys3a phenotype by NIR spectroscopy.

The two lys5 alleles lys5f (Risø mutant 13) and lys5g (Risø mutant 29) were selected by the dye-binding method (Doll (1983), supra) which indicates an increase in basic amino acids such as lysine. The lysine content in these mutants was only slightly increased (10%). Later Greber et al. (2000, Proceedings 8th International Barley Genetics Symposium. Adelaide October 2000. Volume 1, 196-198) suggested that these mutants should be looked upon as having gene lesions in the starch synthesis pathway because they were considerably reduced in starch (50-75% compared to normal barley near isogenic controls).

However, if we compare the sum of starch and BG content of these mutants the picture changes (Table IA). lys5g now even seems to exceed normal barley (58.0 versus 53.9%), and even for the extreme mutant lys5f the low starch content (29.8%) is compensated for with a high BG content (19.8%) to give a total starch and BG content as high as 49.4%. As far as we know such BG compensating effects of starch reducing genes have not been found before.

It was thus surprising to note (Table IA) that the lys5 cluster in FIG. 1B, in addition to a low level of starch, is characterised by very high BG levels fully or partially compensating for the decrease in starch. The extreme gene lys5f produces BG-levels as high as 19.8% compared to 13.3% for lys5g and a value of 6.5% for normal barley. Although a greenhouse environment regularly produces a higher BG and protein content than in the field (compare Tables IA with IB), the effect of the lys5 genes on BG is spectacular.

The allele lys3m (induced in Minerva) originally selected as a low BG mutant at Carlsberg (Munck (1992), supra) has an extremely low A/P index of 9.5 compared to 17.5 in Minerva. It is interesting to note that the mutant allele lys3c in Bomi differs from the other alleles in displaying a normal barley BG value of 6.1%. The other lys3 alleles are all low in BG (approximately 2.5%). The double recessive lys3a5g has an A/P index and BG content intermediate between lys3a and lys5g barley verifying its intermediate position in the NIR classification by PCA between the lys3 and lys5 classes (FIG. 1B).

TABLE IA
CHEMICAL PROPERTIES OF THE 54 SAMPLES (GROUP 1) AND THE
SIX ORIGINAL MUTANTS (GROUP 2) GROWN IN A GREENHOUSE.

| Green | | | Starch (S) | | | | | | Lys | Glu |
|---|---|---|---|---|---|---|---|---|---|---|
| house | n | BG (%) | (%) | BG + S | Protein (%) | Amide (%) | A/P | Fat (%) | (mol %) | (mol %) |
| lys3a | 3 | 4.73 ± 0.98 | 40.4 ± 1.0 | 45.2 ± 0.1 | 17.7 ± 0.9 | 0.32 ± 0.03 | 11.41 ± 0.56 | 3.51^{a} | 4.94^{a} | 14.86^{a} |
| lys3b | 2 | 3.05 ± 0.78 | — | — | 17.1 ± 0.7 | 0.32 ± 0.01 | 11.51 ± 0.22 | — | — | — |
| lys3c | 2 | 6.10 ± 1.56 | — | — | 17.5 ± 0.5 | 0.34 ± 0.01 | 11.96 ± 0.93 | — | — | — |
| lys3m | 2 | 2.25 ± 0.01 | 39.3 ± 1.3 | 41.6 ± 1.3 | 17.4 ± 0.5 | 0.27 ± 0.01 | 9.50 ± 0.06 | — | — | — |
| lys5f | 3 | 19.80 ± 0.20 | 29.8 ± 0.6^{b} | 54.8 ± 6.0^{b} | 17.0 ± 1.4 | 0.42 ± 0.06 | 15.52 ± 0.99 | 3.69^{a} | 3.32^{a} | 27.59^{a} |
| lys5g | 6 | 13.26 ± 0.56 | 44.7^{d} | 58.2^{d} | 17.4 ± 1.0 | 0.43 ± 0.04 | 15.46 ± 0.48 | 2.30 ± 0.25^{a} | 3.76^{a} | 20.09^{a} |
| 3a5g | 3 | 7.8 ± 1.3 | 34.7 ± 10.5^{b} | 43.2 ± 10.6^{b} | 17.2 ± 1.7 | 0.37 ± 0.02 | 13.6 ± 1.1 | — | 4.0^{a} | 20.1^{a} |
| Normal | 33 | 6.45 ± 2.67^{a} | 47.8 ± 1.0^{c} | 54.3 ± 1.8^{c} | 16.2 ± 1.3 | 0.44 ± 0.01 | 19.95 ± 0.62 | 1.94 ± 0.16 | 3.05 ± 0.15 | 24.13 ± 0.83 |
| incl. B | | | | | | | | | | |
| Bomi | 1 | 6.80 | 48.8 | 55.6 | 14.6 | 0.38 | 16.24 | 1.74 | 3.27 | 22.90 |
| (B) | | | | | | | | | | |
| lys4d | 1 | 4.0 | 41.1 | 45.1 | 17.5 | 0.37 | 13.21 | — | 4.04 | 19.46 |
| 16 | 2 | 16.6 ± 1.9 | 29.9^{a} | 45.1^{a} | 17.1 ± 1.5 | 0.45 ± 0.06 | 16.26 ± 0.92 | — | 3.37^{a} | 22.50^{a} |
| 449 | 2 | 13.5 ± 0 | 26.5^{a} | 40.0^{a} | 20.7 ± 2.3 | 0.05 ± 0.06 | 15.14 ± 0.02 | — | — | — |
| w1^{e} | 1 | 15.4 | 27.3 | 42.7 | 16.5 | 0.40 | 15.14 | — | — | — |
| w2^{e} | 1 | 7.0 | 49.0 | 56.0 | 17.4 | 0.47 | 16.93 | — | — | — |

TABLE IB
CHEMICAL PROPERTIES OF 18 SAMPLES FROM GROUP 1 AND 7 SAMPLES GROUP 2, FIELD GROWN.

| | | | Starch (S) | | | | | | Lys | Glu |
|---|---|---|---|---|---|---|---|---|---|---|
| Field | n | BG (%) | (%) | BG + S | Protein (%) | Amide (%) | A/P | Fat (%) | (mol %) | (mol %) |
| lys3a | 1 | 3.1 | 48.5 | 51.6 | 12.7 | 0.23 | 11.36 | 2.63 | — | — |
| lys3m | 1 | 2.4 | 48.8 | 51.2 | 12.3 | — | — | — | — | — |
| lys5f | 1 | 16.5 | — | — | — | — | — | 3.77 | 3.8 | 19.9 |
| lys5g | 2 | 8.9 ± 1.0 | — | — | 11.8 ± 0.1 | 0.26^{d} | 13.7^{d} | — | — | — |
| 3a5g | 1 | — | — | — | 15.5 | 0.28 | 11.3 | — | 4.8 | 15.6 |
| Normal | 13 | 4.5 ± 0.8 | 55.1 ± 2.2 | 49.7 ± 5 | 11.3 ± 1.1 | 0.28 ± 0.04^{g} | 15.4 ± 0.6^{g} | 1.90 ± 0.21 | — | — |
| incl. B | | | | | | | | | | |
| Bomi | 1 | 4.9 | 53.6 | 58.5 | 11.5 | 0.29 | 16.4 | 1.91 | 3.5 | 21.8 |
| (b) | | | | | | | | | | |
| lys4d | 1 | 4.1 | — | — | 12.9 | 0.29 | 14.0 | — | 4.2 | 19.1 |
| 16 | 1 | 12.0 | — | — | 13.8 | 0.32 | 14.5 | — | — | — |
| 95^{f} | 2 | 12.2 | 29.6^{d} | 41.8^{d} | 15.1 ± 0.5 | 0.35 ± 0.03 | 14.2 ± 0.7 | — | — | — |
| 449 | 1 | 12.4 | — | — | 14.6 | 0.32 | 13.7 | — | — | — |
| w1 | 1 | 15.6 | — | — | — | — | — | — | — | — |
| w2 | 1 | 5.7 | — | — | 13.0 | 0.33 | 15.9 | — | — | — |

^{a}n = 30,
^{b}n = 2,
^{c}n = 9,
^{d}n = 1,
^{e}content of amylose: w1 = 20.3% and w2 = 4.2% of starch,
^{f}one sample grown in outdoor pots,
^{g}n = 11

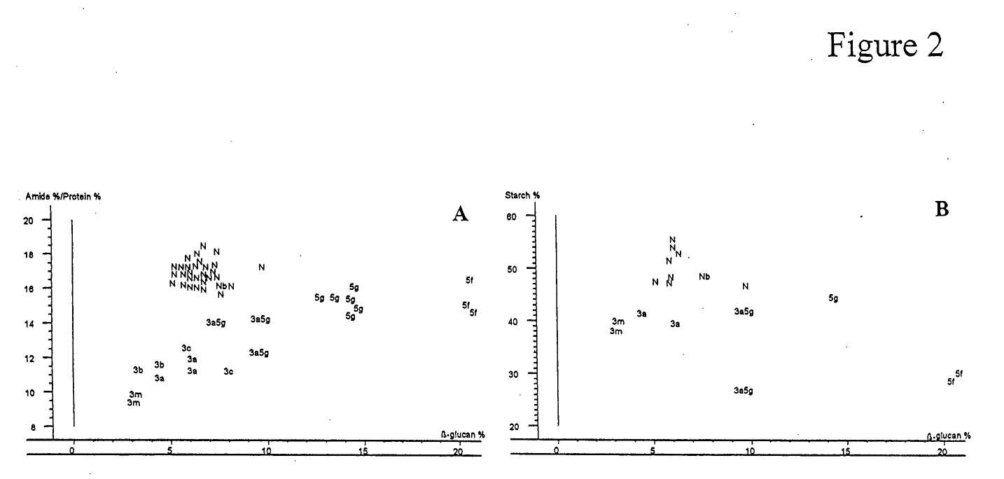

As is discussed by Jacobsen et al. (2004, supra) there are many chemical ways to detect barley mutants because they give a range of specific complex physical-chemical imprints on the phenotype only detectable as a whole by multivariate pattern recognition analysis. It is surprising to note that very simple chemical plots and ratios such as the BG (abscissa) and A/P index (ordinate) in FIG. 2A and even starch (abscissa) and BG (ordinate) in FIG. 2B suffice for successful gene classification as compared to the PCA of corresponding NIR data in FIG. 1B. The efficiency of simple ratios and plots in mutant classification is discussed by Munck (1972), Hereditas 72, 1-128 (pages 79-80).

The NIR approach is useful because the screening and classification can be performed empirically on unknown material picking up a broad physical-chemical fingerprint of the endosperm phenome, also including unexpected effects such as BG levels. The spectra can be interpreted by PCA representing the total effects of genetic covariance (pleiotropy and linkage) of the mutant gene preferably compared against a near isogenic background with barley material grown in the same environment. This involves not only the detection of chemical bonds of obvious interest (here from amide, starch and BG) but also the important indirect physical effects of the genes e.g., of importance for the granulation of the barley flour which may be registered by NIR in spite of multiple scatter correction (MSC). The indicative wavelengths for chemical bonds can be found in the spectroscopic literature (Osborne et al. (1993), supra) giving a hint of which chemical analyses that should be performed for validation of the supervised NIR classification.

In FIG. 1C five wavelengths for specific absorbers indicative for starch, amino acid, cellulose (2) and unsaturated fat are selected from the many possible in the area 2270-2380 nm in order to characterise specific spectral areas which show significant differences.

The NIR approach also picks up unexpected correlations such as with water content, for which NIR spectroscopy is very sensitive. Thus, the high BG content at the expense of starch in lys5 seems to result in a higher content of dry matter (and lower content of water) by approximately 1.5% (Table IA-B). This is presumably due to more molecular water being bound to crystalline starch in the amyloplasts compared to water bound to BG in the endosperm cell wall. Thus the specific effect of water associated with the lys5 gene is also included in the spectral classification together with a broad range of other side effects from the mutant gene.

These pleiotropic effects are automatically summed up in the gene specific spectral fingerprint by a PCA which can be chemically and physically defined a posteori after measurement.

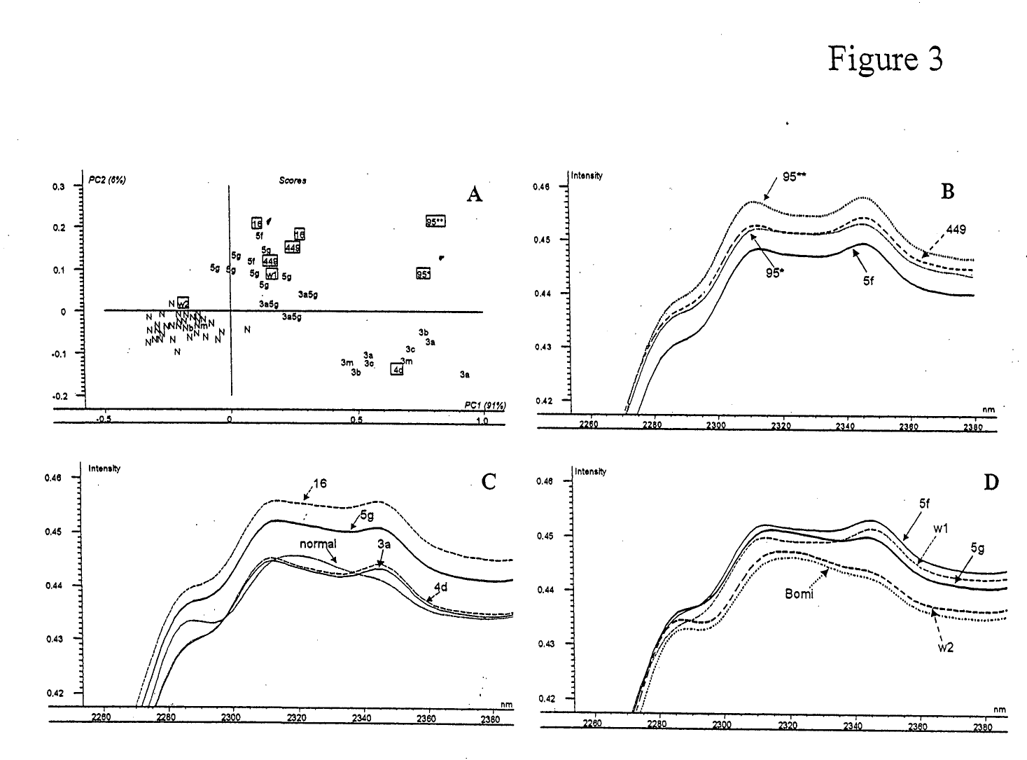

Nine samples from the six new genotypes in group 2 were measured by NIR spectroscopy and added to the 54 spectra (FIG. 1A) in a new PCA with 63 samples (FIG. 3A). Seven of the barley samples were grown in a greenhouse. The two samples of mutant 95 were grown in the field and outdoor in pots. Mutant 16, mutant 449 and w1 (noted as a waxy line) were all located in the BG rich cluster around lys5 with the two mutant 95 samples above to the right.

w2 (also considered as waxy) was included in the upper part of the normal cluster closer to the lys5 area, while the lys4d barley sample resides in the very high lysine lys3 area below to the right. In FIG. 3B it can be seen that mutants 95 and 449 show a resemblance in the 2270-2380 nm region lys5f because they have similar scores, hence similar spectral profiles. The two mutant 95 samples grown in the field and outdoors in pots have similar profiles but are shifted above the baseline due to the environmental difference. Mutant 16 in Bomi has a spectral form in the 2270-2380 nm area which resembles lys5g as seen in FIG. 3C. As expected from the classification in FIG. 3A the spectrum of lys4d (also mutant in Bomi) has a similar form to that of lys3a (FIG. 3C) indicating a major change in amino acid composition. w1 in the lys5 cluster has a spectral form resembling that of lys5f, while w2 is near to that of normal barley (Bomi) (FIG. 3D).

The result of the chemometric classification analysis of the spectral information in FIG. 3A is validated by the chemical analyses shown in Tables IA and IB. All the new mutants positioned in the lys5 cluster (or near to it, such as mutant 95) have strongly increased levels of BG (individual samples), namely mutant 16 (15.2%), mutant 449 (13.5%), and w1 (15.4%). The starch contents of the new mutants were lower than those of lys5g and lys5f, and the starch plus BG level (% d.m) were clearly below the normal lines, as they were for the lys5g and lys5f mutants (Table IA). Also mutant 95 grown outdoors in the field and in pots is on the high BG side above the baseline in the PCA in FIG. 3A. It is high in BG (12.2 and 14.2%). It is clear that the position in the PCA plot has been altered because of environmental effects. Since its spectral form is near to that of lys5f (FIG. 3B), it should belong to the lys5 cluster in the PCA score plot in FIG. 3B if grown in a greenhouse.

lys4d, which was classified in the lys3 cluster with a changed amino acid pattern and low BG content, is confirmed to have a low A/P index and BG as is the case with lys3a, lys3b and lys3m (Table IA and IB). This is further verified by the amino acid composition (lysine and glutamine/glutamic acid) relative to Bomi in Table IA, which is significantly changed in the direction of lys3a in lys4d.

In evaluating the two supposedly waxy mutants (Table IA and IB) it can be seen that w1 has a practically normal amylose content of 20.3% but is very high in BG (15.4%) and low in starch (42.7%). Therefore w1 is not a classic waxy high amylopectin low amylose mutant with slightly increased BG, but rather a mutant in the new category of low starch/high BG mutants. w2 which has a NIR spectral form (FIG. 3D) closer to normal barley (Bomi) is waxy. It has a low amylose content (4.2%) and a BG content on the high side (7.0%) compared to normal barley in Table IA (mean 6.5%).

With a synergistic combination of spectroscopic and chemometric tools employed on cereal seeds, it has thus been possible to detect previously unknown endosperm genes and mutants. In such a study it is difficult to differentiate between the two different sources of covariance, pleiotropy (biochemical gene applications) and linkage (association of adjacent genes on the chromosome). We, therefore name the combined effect of pleitropy and linkage “genetic covariance”. This technology allows a truly exploratory strategy, with a minimum of hypotheses, where the chemical effects of the gene are determined after selection using PCA with the spectra as preliminary guidelines (Osborne et al. (1993), supra) for generating new hypotheses in a dialogue with a priori knowledge.

High to moderate levels of BG and a high content of free sugars and even phytoglycogens are, in many mutant alleles, associated with the amylopectin waxy gene (wax) in barley (Newman and Newman (1992), Nutritional aspects of barley seed structure and composition, in: Shewry, (ed.), Barley: Genetics, Molecular Biology and Biotechnology, CAB International, Wallingford, U.K. 351-368; Fujita et al. (1999), Breeding Science 49, 217-219). There are only slight reductions in starch level and seed size. It is interesting to note that the lys5g (mutant 29) and mutant 16 found here to be high in BG have approximately normal levels of amylose (Tester et al. (1993), supra). The sum of starch and BG in these mutants, as shown in Tables IA and IB, however, approaches normal values in percentage of dry matter with lys5g as the best performer. In addition, the high lysine amino-acid mutants lys3a and lys4d in our study, displayed a reduction in BG but are unchanged in amylose levels (Tester et al. (1993), supra).

The BG content of the six BG compensated starch reduced barley mutants reported here is extremely high compared to the results reported in review given e.g., by MacGregor and Fincher (1993, in: MacGregor & Bhatty (eds.), Barley—Chemistry and Technology, American Association of Cereal Chemists St. Paul, 247) finding a range of 2.8-10.7% d.m.

There are amylose free waxy (wax) genes as well as alleles which contain amylose such as the w2 line (4.2% apparent amylose) reported in this investigation (BG 7.0%, see Table IA). The high amylose amo 1 barley genes such as in Glacier AC38 (apparent amylose 40.6%) also have a moderately increased content of BG (7.9%) compared to a normal variety (BG 4.7%, apparent amylose 33.1%) as reported by Fujita et al. (1999, supra). Swanston et al. (1995, J. Cereal Sci. 22, 265-273) demonstrated that the double recessive line between a waxy (non amylose free) and the amo 1 genes had 9.4% in BG compared to 3.6% for the controls. This was confirmed by Fujita et al., 1999 (supra) with a waxy amylose free allele combined with the gene amo 1 in a double recessive line that reached the high BG level of 12.4%, about 2.6 times higher than the control line. In this paper the six BG compensated starch mutants have a range in BG of 8.9-16.5% when grown in the field and 13.3-19.8% when grown in a greenhouse compared to a mean of 4.5% and 6.5% respectively for a set of control varieties (Tables IA and IB). This change in BG amounts to 2.0 to 3.7 times.

High lysine mutants such as mutants lys3a,b,m and lys4d seem to have reduced BG contents (Table IA). Note that the allele in the lys3 locus lys3c has a normal BG content. This fact suggests that the high lysine and BG reducing traits here are controlled by adjacent genes and that the mutations involve chromosome segments of different lengths around the lys3 locus in chromosome 5. There is a positive significant correlation between the A/P index and the BG content of lys3 genotypes of r=0.83, which indicates a position effect of the different alleles. The reduced BG content should thus not be pleiotropic to the lys3a, lys3b and lys3m alleles but rather depend on a very tight linkage (which is very difficult to break by recombination) to an adjacent gene, which retards BG synthesis in three of the four lys3 genotypes. According to our experience BG synthesis is active, also rather late in kernel development and is dependent on environmental factors such as heat and precipitation (Aastrup (1979), Carlsberg Research Communications 44, 381-393). It is inherited in normal barley by a simple genetic additive system (Powell et al. (1985), Theor. Appl. Gen. 71, 461-466). Although the BG content is higher under greenhouse conditions, the environmental differences between most mutants and normal barley are consistent (compare Table IA with IB).

Thus, NIR spectroscopy may be used as a screening method, based on the relationship between starch and BG synthesis. Genes that regulate BG synthesis appear to be closely coupled to and apparently compete with those genes that regulate starch synthesis in the developing endosperm. The sugar precursors available are shifted in their destiny accordingly. Three of the six BG compensating starch mutants are confirmed to have a normal starch amylose to amylopectin composition. At least two gene loci lys5 (alleles lys5f and lys5g) in chromosome 6 and mutant 16 in chromosome 7 are involved in the BG compensated starch mutant trait.

### CONCLUSION

In this investigation we have classified by NIR spectroscopy ten of the classic “high lysine” barley mutants of the 20-30 available. We have found that five of those genes—the lys5f, lys5g alleles in chromosome 6, mutant 16 in chromosome 7 (Doll (1983), supra) and mutants 95 and 449 (Di Fonzo and Stanza (1977), supra) with unknown chromosome locations—combine low starch synthesis with excessive BG synthesis completely or partly compensating for the decrease in starch formation. Two of the five high BG compensating starch mutants—Risø mutants 16 and lys5g originally selected as high lysine have proved to have normal amylose content by Tester et al. (1993), supra. Additionally a sixth high BG, low starch mutant content was found (w1; 1201) which previously had been selected falsely by plant breeders as waxy. It has a normal amylose content. The presence of high BG levels in barley, reported previously in the literature, has been mainly associated with either low (waxy) or high amylose (amo 1) genes. To our knowledge, data combining normal amylose barley with BG levels approaching 20% (lys5f, Table IA) have not been published before.

The introduction of spectroscopy and chemometrics makes it possible to reveal specific gene expression patterns as discussed here and by Munck et al. (2001, supra) and Munck (2003, supra) on the level of the phenome. Chemometric pattern recognition statistical methods e.g., through PCA is now starting to be used more frequently in molecular biology to connect different levels of biological organisation (Fiehn (2002), Plant Molecular Biology 48, 155-171). NIR spectroscopy as demonstrated in this paper and other spectroscopic screening methods such as Nuclear Magnetic Resonance (NMR) evaluated by chemometrics should be effective in revealing new metabolic mechanisms.

This technology allows for the rapid screening and identification of seed on the basis of e.g., spectral properties to identify those with desired characteristics, in this case low starch and high BG.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A method of preparing a seed product comprising the step of subjecting one or more seeds having low starch and high β-glucan to one or more processing steps.

2. The method of claim 1 wherein said starch is 25-40% of the seed, dry weight.

3. The method of claim 1 wherein said β-glucan is 10-25% of the seed, dry weight.

4. The method of claim 1 wherein said starch comprises 8-35% amylose and the remainder is provided by amylopectin.

5. The method of claim 1 wherein said product is selected from the list consisting of meal, flour and flakes.

6. The method of claim 1 wherein said seeds are from Risømutants 13, 16, 29, Perga mutants 95, 449 or waxy line w1.

7. The method of claim 1 wherein said seed is modified relative to the wild-type seed.

8. The method of claim 7 wherein an existing endogenous gene is modified or mutated or exogenous nucleic acid material is added.

9. The method of claim 8 wherein said seed is derived from plants or plant cells which have been transfected with sense nucleic acid molecules comprising an unmodified, modified or mutant sequence or with an antisense sequences to the wild-type sequence to impair expression of the wild-type sequence.

10. The method of claim 8 wherein said mutation is in the lys5 locus in chromosome 6 or in chromosome 7.

11. The method of claim 8 wherein the sequence which is modified or mutated is brittle-1 (Accession number AY033629) α-glucosidase (Accession No. AAF76254.1) or 3-glucanase (Accession No. AAL73976.1).

12. The method of claim 7 wherein one or more of the genes encoding at least one of the AGPase components is modified, preferably mutated, such that the seed exhibits lower levels of AGPase or lower levels of AGPase activity relative to wild-type.

13. The method of claim 12 wherein the sequence which is modified or mutated is the AGPaseS1 gene, preferably from barley, wheat, maize or rice.

14. The method of claim 13 wherein the gene which is modified or mutated comprises the sequence of SEQ ID NO: 1,
or a portion thereof,
or a sequence which hybridizes to said sequence or portion thereof under non-stringent binding conditions of 6×SSC/50% formamide at room temperature and washing under conditions of high stringency,
or a sequence which exhibits at least 80% sequence identity to said sequence or portion thereof,
or a sequence complementary to any of the aforesaid sequences.

15. A seed product obtainable by the method of claim 1.

16. The seed product of claim 15, wherein said seed product is β-glucan which is isolated from said seed.

17. A method of preparing a seed having low starch and high β-glucan, comprising the steps of:
a) inserting an exogenous nucleic acid sequence into one or more plant cells; wherein said nucleic acid sequence is selected from sense nucleic acid molecules comprising unmodified, modified or mutant sequences, and antisense sequences to a wild-type sequence to impair expression of the wild-type sequence; and
b) obtaining or propagating a seed therefrom.

18. A method of screening for seeds having low starch and high β-glucan comprising the steps of:
a) determining one or more phenotypic characteristics of one or more positive seed standards with low starch and high β-glucan;
b) determining said one or more phenotypic characteristics of one or more negative seed standards;
c) generating a fingerprint representation of the results of said phenotypic characteristics determined in steps a) and b), wherein said fingerprints for said positive and negative seed standards are separable;
d) determining said one or more phenotypic characteristics of a test seed and generating a fingerprint representation using the method of step c);
e) comparing the fingerprint generated in step d) with the fingerprints generated in step c), wherein correlation of the fingerprint to the positive or negative seed standard is indicative of the presence or absence of low starch and high β-glucan, respectively; and
f) optionally propagating the seed having low starch and high β-glucan for one or more generations.

19. A method of screening for seeds having low starch and high β-glucan comprising the steps of:
a) performing Near Infrared Reflection spectroscopy on one or more positive seed standards with low starch and high β-glucan to generate spectral traces for said standards;
b) performing Near Infrared Reflection spectroscopy on one or more negative seed standards to generate spectral traces for said standards;
c) performing Near Infrared Reflection spectroscopy on a test seed to generate a spectral trace for said test seed,
d) comparing the spectral trace generated in step c) with the spectral traces generated in steps a) and b), wherein correlation of the trace to the positive or negative seed standards is indicative of the presence or absence of low starch and high β-glucan, respectively; and
e) optionally propagating the seed having low starch and high β-glucan for one or more generations.

20. The method of claim 19 wherein said spectroscopy is performed at one or more of the following wavelengths: 1100-1400 nm, 1400-1800 nm, 1800-2500 nm, 1890-1920 nm, and/or 2260-2380 nm.

21. A seed identified by the screening method of claim 18 or 19.

22. A seed having low starch and high β-glucan, wherein said seed has been produced by adding exogenous nucleic acid material to said seed or plant or plant cells used to generate said seed.
